(19) United States
(12) Patent Application Publication
(10) Pub. No.: US 2014/0012024 A1
(43) Pub. Date: Jan. 9, 2014

(54) METHOD FOR THE SYNTHESIS OF DHA

(71) Applicants: Mohamed Amin Khan, Morgan Hill, CA (US); Paul L. Wood, Harrogate, TN (US)

(72) Inventors: Mohamed Amin Khan, Morgan Hill, CA (US); Paul L. Wood, Harrogate, TN (US)

(73) Assignee: PHENOMENOME DISCOVERIES INC., Saskatoon, SK (CA)

(21) Appl. No.: 14/006,717

(22) PCT Filed: Mar. 16, 2012
(86) PCT No.: PCT/CA12/00239

§ 371 Date: Sep. 23, 2013

Related U.S. Application Data
(60) Provisional application No. 61/466,606, filed on Mar. 23, 2011.

(51) Int. Cl. C07C 51/09; C07C 303/28; C07C 67/317; C07C 69/606

(52) U.S. Cl. CPC C07C 51/09; C07C 69/606; C07C 303/28; C07C 67/317
USPC 554/154; 554/224; 558/56

(57) ABSTRACT

A method for preparing docosahexaenoic acid (DHA). The method comprises coupling a compound represented by Formula I with a compound represented by Formula II followed by partial hydrogenation to obtain a compound represented by Formula III. The compound represented by Formula III acts as a DHA precursor and thus can be hydrolysed to obtain DHA. Novel starting materials represented by Formula I and Formula II, and synthetic routes for preparing the same are also provided.

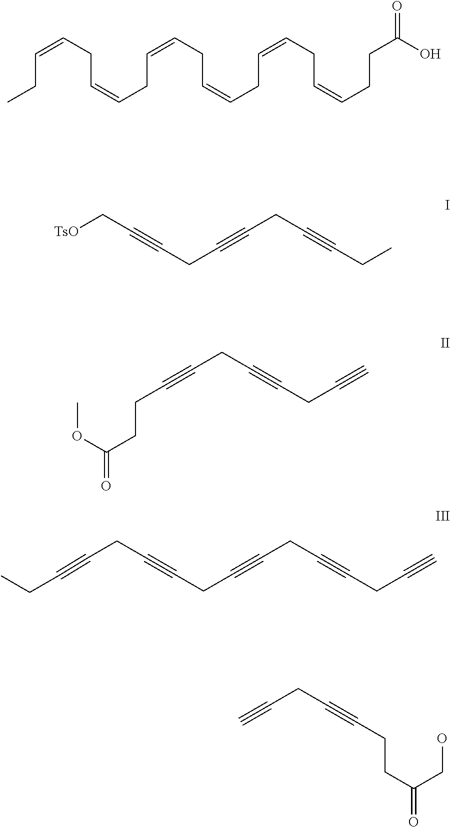

### FIELD OF INVENTION

The present invention relates to the field of synthetic chemistry. Specifically, the present invention relates to chemical syntheses of DHA, and more specifically, to starting materials, precursors and methods for the chemical synthesis of DHA on industrial scale.

### BACKGROUND OF THE INVENTION

Docosahexaenoic acid (DHA) is an omega-3 unsaturated fatty acid, containing a chain-terminating carboxylic acid group and six cis-double bonds in a 22-carbon straight chain. Its trivial name is cervonic acid, its systematic name is all-cis-docosa-4,7,10,13,16,19-hexa-enoic acid, and its shorthand name is 22:6w3 in the nomenclature of fatty acids. Its chemical structure can be represented as follows:

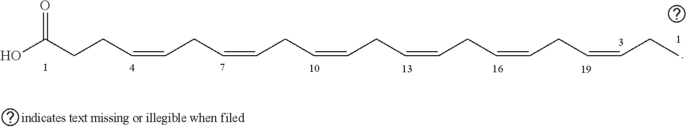 [SMILES: CC/C=C\C/C=C\C/C=C\C/C=C\C/C=C\C/C=C\CCC(=O)O]

It is derived from the essential precursor linolenic acid (LNA, 18:3w3). DHA is the main end-product of LNA after successive desaturations and elongations, a metabolic cascade that is assumed to be weak in humans [Burdge G C, Jones A E, Wootton S A (2002) Eicosapentaenoic and docosapentaenoic acids are the principal products of alphalinolenic acid metabolism in young men. Br J Nutr 88:355-363; Brenna J T, Salem N Jr, Sinclair A J, Cunnane S C (2009) Alphalinolenic acid supplementation and conversion to n-3 long-chain polyunsaturated fatty acids in humans. Prostaglandins Leukot Essent Fatty Acids 80:85-91].

DHA is essential for the growth, functional development and healthy maintenance of brain function and is required throughout life from infancy through aging [Horrocks, L. A. and Y. K. Yeo. Pharmacol. Res. 40(3):211-225 (1999)]. It is observed that DHA is mainly esterified in membrane phospholipids of the brain, retina, and spermatozoa [Salem N Jr, Litman B, Kim H Y, Gawrisch K (2001) Mechanisms of action of docosahexaenoic acid in the nervous system. Lipids 3:945-959].

DHA has been attributed to physiological effects such as blood lipid reduction, anticoagulant effect, carcinostatic effect, and improvement in visual functions. DHA was found to inhibit growth of human colon carcinoma cells [Kato T, Hancock R L, Mohammadpour H, McGregor B, Manalo P, Khaiboullina S, Hall M R, Pardini L, Pardini R S (2002). “Influence of omega-3 fatty acids on the growth of human colon carcinoma in nude mice”. Cancer Lett. 187 (1-2): 169-77]. Dietary DHA may reduce the risk of heart disease by reducing the levels of blood triglycerides in humans. Further, DHA deficiencies are associated with foetal alcohol syndrome, attention deficit hyperactivity disorder, cystic fibrosis, phenylketonuria, unipolar depression, aggressive hostility and adrenoleukodystrophy. In contrast, increased intake of DHA has been shown to be beneficial or have a positive effect in inflammatory disorders (e.g., rheumatoid arthritis), Type II diabetes, hypertension, atherosclerosis, depression, myocardial infarction, thrombosis, some cancers and for prevention of the onset of degenerative disorders such as Alzheimer's disease (U.S. Pat. No. 7,550,286 B2).

Fish, such as salmon, tuna, and mackerel, have been used as a major source of DHA, since they naturally contain high concentrations of this long-chain fatty acid. More particularly, about 20% of sardine-or tuna-derived oil is made up of DHA (U.S. Pat. No. 7,259,006 B2).

Most of the DHA used in medicine or as a dietary supplement, is separated and purified from DHA-containing fish oil. However, a variety of other polyunsaturated fatty acids having structures similar to that of DHA are contained in fish oil, and thus, separation and purification are difficult. For example, it is preferred to use a DHA-containing fat and oil having a low content of icosapentaenoic acid during preparation of DHA. However, when the source is fish oil, it is very difficult to efficiently remove icosapentaenoic acid only.

Additionally, due to the presence of the polyunsaturated fatty acids such as arachidonic acid (ARA) or icosapentaenoic acid (EPA) (U.S. Pat. No. 7,514,244 B2), DHA is likely to be oxidized. Oxidation of DHA (in general, fatty acids) is known to produce free radicals that may play a role in the development of cancer and other degenerative diseases.

Another major problem in terms of the use of fish-derived DHA-containing fat and oil in the field of food products is the necessity of considerable operations for removing the smell of fish. Also, vegetarians are likely to not accept any food product/drug containing DHA derived from fish oil and hence have to depend on alternative dietary supplements as a source of DHA.

One of the existing methods to overcome the problems in obtaining DHA from fish oil is disclosed by Teshima et al., in Bulletin of the Japanese Society of Scientific Fisheries, 44(8) 927 (1978). In this document, they describe a method for isolating EPA and DHA from squid liver oil by saponifying with ethanolic potassium hydroxide, extracting the fatty acids with ether, and methylating. The crude fatty acid methyl ester is purified by column chromatography on Silica Gel 60, and then the EPA is separated from the DHA by column chromatography on a mixture of silver nitrate and silica gel. However, the problem with this method is that there are often traces of silver left in the final product, which is extremely undesirable in a food or pharmaceutical for human consumption. Furthermore, very high amounts of solvent are necessary to conduct the column chromatography, which in turn increase the costs.

Another known source of DHA is the lipids accumulated in cultured cells of a microorganism having an ability to produce DHA. Numerous different processes exist based on specific microbial organisms utilized e.g., Schizochytrium species (U.S. Pat. Nos. 5,340,742; 6,582,941); Ulkenia (U.S. Pat. No. 6,509,178); Pseudomonas sp. YS-180 (U.S. Pat. No. 6,207,441); Thraustochytrium genus strain LFF1 (U.S. 2004/0161831 A1); Crypthecodinium cohnii [U.S. 2004/0072330 A1; de Swaaf, M. E. et al. Biotechnol Bioeng. 81(6):666-72 (2003) and Appl Microbiol Biotechnol. 61(1):40-3 (2003)]; Emiliania sp. (Japanese Patent Publication (Kokai) No. 5-308978 (1993)); and Japonochytrium sp. (ATCC #28207; Japanese Patent Publication (Kokai) No. 199588/1989)].

Additionally, the following microorganisms are also known to have an ability to produce DHA: Vibrio marinus (a bacterium isolated from the deep sea; ATCC #15381); the micro-algae Cyclotella cryptica and Isochrysis galbana; and, flagellate fungi such as Thraustochytrium aureum [ATCC #34304; Kendrick, Lipids, 27:15 (1992)] and the Thraustochytrium sp. designated as ATCC #28211, ATCC #20890 and ATCC #20891.

However, in using any of the above-mentioned microorganisms, several problems exist, such as a low yield of DHA, a requirement of a prolonged culture period for obtaining a sufficient amount of DHA, or a requirement of a specific medium or culture condition for production.

When an alga such as Emiliania sp. is utilized for the production, a high yield of DHA may be accomplished, however, the culture steps are complicated due to the requirement of light for cultivation. Consequently, such a process is not suitable for industrial production (U.S. Pat. No. 7,514,244 B2).

Fermentation processes also exist for commercial production of DHA, for example, by fermentation of C. cohnii for production of DHASCO™ (Martek Biosciences Corporation, Columbia, Md.); fermentation of Schizochytrium sp. for production of an oil formerly known as DHAGold (Martek Biosciences Corporation); and fermentation of Ulkenia sp. for production of DHActive™ (Nutrinova, Frankfurt, Germany). Despite these successes, each of these methods suffer from an inability to substantially improve the yield of oil or to control the characteristics of the oil composition produced, since the fermentations rely on the natural abilities of the microbes themselves (US 2009/0311380 A1).

Kyle et al., in U.S. Pat. No. 5,397,591, disclose a method for obtaining DHA from cultivation of dinoflagellates in a fermentor, induction of the dinoflagellates to produce single cell oil having a high proportion of DHA, and recovery of that oil. Preferably, the oil recovered contains at least about 20% by weight of DHA, and more preferably, more than about 35% by weight DHA. However, the product recovered is not pure DHA, but a mixture of DHA in other oils, which is difficult to separate.

Another source, which is the most direct and complete source of DHA, is found in the blubber of certain marine mammals, especially the harp seal. One of the prominent advantages of DHA derived from seal blubber is its faster and more thorough absorption by the human body as compared to that of the DHA derived from fish oils. This is due, in part, to the molecular configurations of the DHA in seal oil, which varies slightly from that found in fish oils. However, there are challenges in producing satisfactory grade seal oil for administration as a dietary supplement to humans. Seal oils, like other health food oils, are susceptible to the natural process of oxidation. The primary and secondary products of oxidation may give rise to unacceptable flavors and odors in the oil, impair digestibility of the oil, and produce free radicals which can damage or destroy the body's cells (U.S. Pat. No. 7,179,491 B1).

It has heretofore been very difficult to obtain pure and stable DHA, since the main source of these fatty acids is in the fats and oils of marine animals, fish oils (such as mackerel oil, menhaden oil, salmon oil, capelin oil, tuna oil, sardine oil, or cod oil), marine algae such as Schizochytrium sp., human milk, and vegetable oils, such as linseed oil, from where DHA cannot be obtained due to one or more of the reasons specified in the foregoing discussion.

### SUMMARY OF THE INVENTION

It is therefore an object of the invention to provide new methods to obtain DHA.

It is also an object, in certain embodiments of the invention, to provide methods to prepare DHA that is highly pure and not contaminated with metallic and/or other impurities, and which is stable on storage.

In certain preferred embodiments it is also an object to provide easy, low cost and/or efficient industrial scale production methods for DHA synthesis.

In one aspect of the invention, a compound represented by the Formula I:

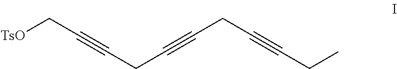 [SMILES: CCC#CCC#CCC#CCOS(=O)(=O)c1ccc(C)cc1]

is provided.

In another aspect of this invention, the use of the compound represented by Formula I as a starting material for synthesis of DHA is provided.

In another aspect of the invention, a compound represented by the Formula II:

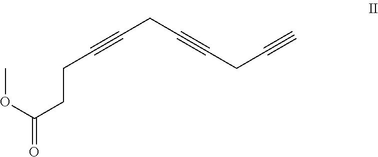

is provided.

In another aspect of this embodiment, the use of the compound represented by Formula II as a starting material for synthesis of DHA is provided.

In yet another aspect of the invention, the use of a compound represented by Formula III:

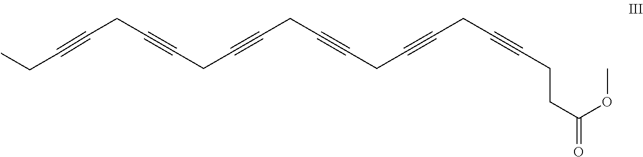 [SMILES: CCC#CCC#CCC#CCC#CCC#CCC#CCCC(=O)OC]

as a precursor for synthesis of DHA is provided.

In another aspect, there is provided a method for preparing a compound represented by Formula I:

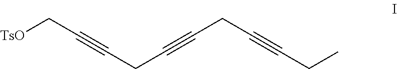 [SMILES: CCC#CCC#CCC#CCOS(=O)(=O)c1ccc(C)cc1]

wherein the method comprises:

(i) reacting propargyl alcohol with an ethyl halide (such as but not limited to ethyl iodide) in the presence of a strong base (such as but not limited to n-BuLi or a secondary-BuLi) and a polar aprotic solvent (such as but not limited to HMPA, THF, MTBE (methyl tert-butyl ether) or a combination thereof to obtain a compound represented by Formula IV:

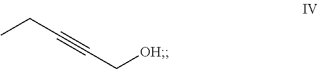

(ii) reacting the compound represented by Formula IV with 4-Toluenesulfonyl chloride (TsCl) and a strong inorganic base (such as but not limited to potassium hydroxide (KOH) sodium hydroxide (NaOH) or lithium hydroxide (LiOH)) to obtain a compound represented by Formula V

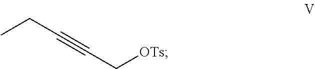 [SMILES: CCC#CCOS(=O)(=O)c1ccc(C)cc1]

(iii) reacting propargyl alcohol with an alcohol protecting group (such as but not limited to DHP, TMS (trimethylsilyl) or TBDMS (tertiary butyl dimethyl silyl) to obtain a compound represented by Formula VIa:

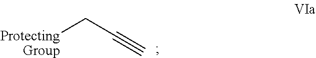

(iv) reacting the compound represented by Formula VIa with a propargyl halide in the presence of a catalytic amount of CuCl to obtain a compound represented by Formula VIIa:

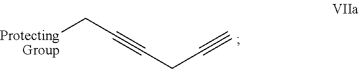

(v) reacting the compounds represented by Formulae V and VIIa the presence of a polar aprotic solvent (such as but not limited to dimethylformamide (DMF), THF or MTBE) to obtain a compound represented by Formula VIIIa:

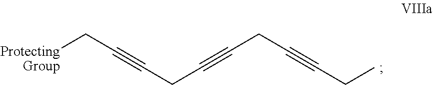

(vi) the protecting group of the compound represented by Formula VIIIa is then removed to obtain the compound represented by Formula IX:

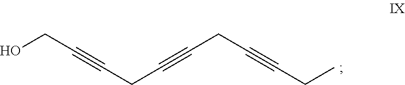

and

(vii) the compound represented by Formula IX is reacted with a tosyl halide (such as but not limited to tosyl chloride) in the presence of pyridine to obtain the compound represented by Formula I.

In certain preferred embodiments of the above-described method, which are not to be considered limiting in any way, a first batch of propargyl alcohol is initially reacted with ethyl iodide in the presence of n-BuLi, HMPA in THF, and at a temperature of between −78° C. to room temperature, to obtain a compound represented by Formula IV. The compound represented by Formula IV is then reacted with TsCl and KOH to obtain a compound represented by Formula V, with an approximate yet non-limiting yield in the range of about 33-40%. In a parallel reaction scheme, DHP protection of a second batch of propargyl alcohol is carried out to prepare a magnesium acetylide compound represented by Formula VI:

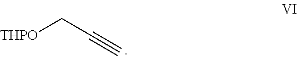

The magnesium acetylide of the compound represented by Formula VI is reacted with propargyl bromide in the presence of CuCl to obtain a compound represented by Formula VII

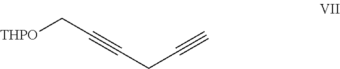

with an approximate yet non-limiting yield ranging from about 65-75%. In the next step, the compounds represented by Formulae V and VII are coupled in the presence of DMF to obtain a compound represented by Formula VIII

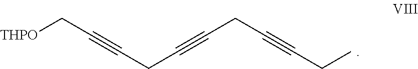

The compound represented by Formula VIII is then, in a non-limiting embodiment, deprotected in the presence of TSA/methanol, and at a temperature of 60° C. to obtain the compound represented by Formula IX with an approximate yet non-limiting yield of 50%. Finally, the compound represented by Formula IX can be reacted with tosyl chloride in the presence of pyridine to obtain the desired compound represented by Formula I. The approximate yet non-limiting yield for compound represented by Formula I according to this embodiment ranges from about 60-65% for this final step.

In another aspect, the invention provides a method for preparing a compound represented by Formula II:

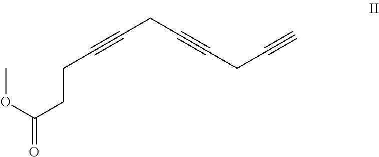

wherein the method comprises:

(i) reacting 2-butyne-1,4-diol with tosyl chloride and pyridine in an organic solvent (such as, for instance, dichloromethane) to obtain a compound represented by Formula X:

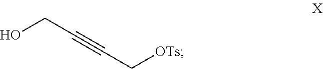 [SMILES: Cc1ccc(S(=O)(=O)OCC#CCO)cc1]

(ii) the compound represented by Formula X is then reacted with trimethylsilyl acetylene to obtain a compound represented by Formula XI

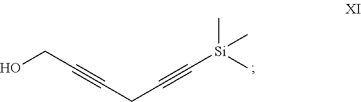

(iii) the compound represented by Formula XI is then tosylated (for example by reacting with p-toluenesulfonic acid) to obtain a compound represented by Formula XII

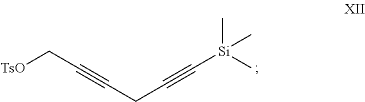 [SMILES: Cc1ccc(S(=O)(=O)OCC#CCC#C[Si](C)(C)C)cc1]

(iv) the compound represented by Formula XII is then reacted with methyl-4-pentynoate to obtain a compound represented by Formula XIII:

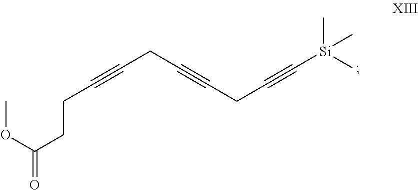

and

(v) the compound represented by Formula XIII is then deprotected (for example, but not limited to reacting with Tetra-n-butylammonium fluoride (TBAF) in a solvent such as dichloromethane) to obtain the desired compound represented by Formula I.

In certain preferred embodiments of the above-described method, which are not to be considered limiting in any way, 2-Butyne-1,4-diol is reacted with tosyl chloride and pyridine in dichloromethane to obtain a compound represented by Formula X with an approximate yet non-limiting yield ranging from about 45-50%. Then, the compound represented by Formula X is reacted with trimethylsilyl acetylene to obtain a compound represented by Formula XI with an approximate yield ranging from about 60-65%. In the next step, the compound represented by Formula XI is tosylated to obtain a compound represented by Formula XII with an approximate yield ranging from about 65-75%. The compound represented by Formula XII is then reacted with methyl-4-pentynoate to obtain a compound represented by Formula XIII with an approximate yield ranging of about 45% in certain embodiments. The compound represented by Formula XIII is then deprotected with TBAF/Dichloromethane to obtain the compound represented by Formula II.

In yet another aspect, the invention provides a method for preparing docosahexaenoic acid (DHA):

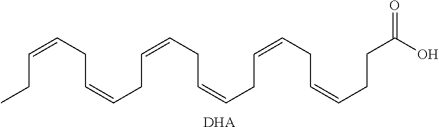

wherein the method comprises:

(i) reacting a compound represented by Formula I

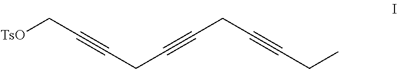 [SMILES: CCC#CCC#CCC#CCOS(=O)(=O)c1ccc(C)cc1]

with a compound represented by Formula II

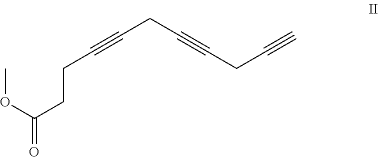

in the presence of CuI, NaI and K₂CO₃ and in a polar aprotic solvent (such as but not limited to dimethylformamide (DMF)), to obtain a compound represented by Formula III:

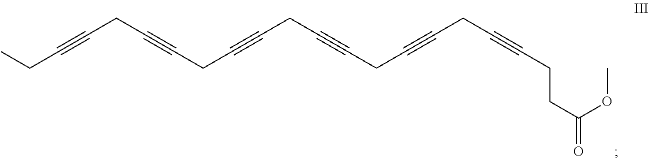 [SMILES: CCC#CCC#CCC#CCC#CCC#CCC#CCCC(=O)OC]

(ii) partially hydrogenating the compound represented by Formula III in the presence of a catalyst (such as, but not limited to Lindlar's catalyst) to produce an ester represented by Formula XIV:

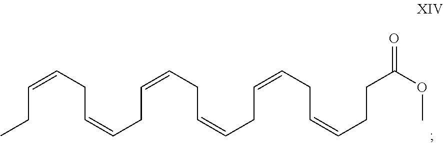 [SMILES: CC/C=C\C/C=C\C/C=C\C/C=C\C/C=C\C/C=C\CCC(=O)OC]

and

(iii) hydrolyzing the ester represented by Formula XIV to produce DHA.

In certain preferred embodiments of the above-described method, which are not to be considered limiting in any way, a compound represented by Formula I can be coupled, in the presence of CuI, NaI and K₂CO₃ in DMF, with a compound represented by Formula II to obtain a compound represented by Formula III with an approximate yield ranging from about 30-40%. Partial hydrogenation of the compound represented by Formula III in the presence of Lindlar's catalyst yields an ester represented by Formula XIV, and the hydrolysis of the ester represented by Formula XIV yields DHA.

In yet another aspect, the invention provides a further method for preparing docosahexaenoic acid (DHA):

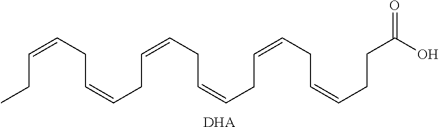

wherein the method comprises:

(i) partially hydrogenating a compound represented by Formula III

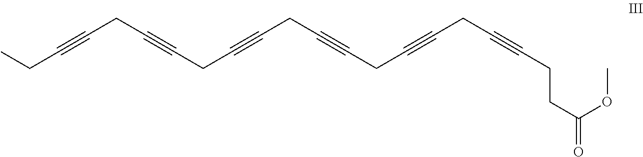 [SMILES: CCC#CCC#CCC#CCC#CCC#CCC#CCCC(=O)OC]

in the presence of a catalyst (such as, but not limited Lindlar's catalyst) to produce an ester represented by Formula XIV

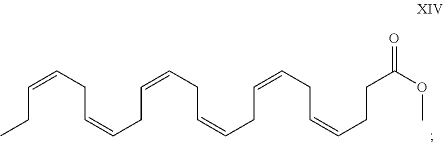 [SMILES: CC/C=C\C/C=C\C/C=C\C/C=C\C/C=C\C/C=C\CCC(=O)OC]

and

(ii) hydrolysing the ester represented by Formula XIV to produce DHA.

### BRIEF DESCRIPTION OF THE DRAWINGS

These and other features of the invention will become more apparent from the following description in which reference is made to the appended drawings wherein:

FIG. 1 shows the ¹H NMR of compound PHR-101;

FIG. 2 shows the ¹H NMR of compound PHR-102;

FIG. 3 shows the ¹H NMR of compound PHR-201;

FIG. 4 shows the ¹H NMR of compound PHR-106;

FIG. 5 shows the ¹H NMR of compound PHR-107;

FIG. 6 shows the ¹H NMR of compound PHR-108;

FIG. 7 shows the ¹H NMR of compound PHR-109;

FIG. 8 shows the ¹H NMR of compound PHR-103;

FIG. 9 shows the ¹H NMR of compound PHR-104;

FIG. 10 shows the ¹H NMR of compound PHR-110;

FIG. 11 shows the ¹H NMR of compound PHR-111;

FIG. 12 shows the ¹H NMR of compound PHR-112;

FIG. 13 shows the ¹H NMR of compound PHR-114;

FIG. 14 shows the ¹H NMR of compound PHR-115; and

FIG. 15 shows the ¹H NMR of compound PHR-116 (DHA).

### DETAILED DESCRIPTION

The present invention provides a novel synthetic route for preparing DHA. Some of the advantages obtained according to certain preferred embodiments of the synthetic route include the ability to prepare highly pure DHA, reduced process costs, abundant and/or inexpensive reagents, and little or no requirement for downstream processing. Further, the high purity of the DHA prepared according to these preferred embodiments of the invention may allow for lowered amounts of DHA product to be used in certain applications, which has the effect of further reducing the cost. Additionally, novel intermediate compounds are provided and which can be used in the synthetic methods described herein.

It will be appreciated by those skilled in the art that each of the embodiments of the instant invention described hereunder may be utilized individually or combined in one or more manners different that the ones disclosed above to produce an improved process for the production of DHA. One skilled in the art will also be able to select a suitable temperature in view of the reaction conditions being used in different aspects and embodiments of the present invention described hereunder.

The patent and scientific literature referred to herein establishes knowledge that is available to those with skill in the art. Unless otherwise defined, all technical and scientific terms used herein have the same meaning as commonly understood by one of ordinary skill in the art to which this invention relates. The issued patents, applications, and references that are cited herein are hereby incorporated by reference to the same extent as if each was specifically and individually incorporated by reference.

Although methods and materials similar or equivalent to those described herein can be used in the practice or testing of the present invention, the preferred methods and materials are described herein. In the case of inconsistencies, the present disclosure, including definitions, will control. In addition, it is to be understood that the materials, methods, and examples are illustrative only and are not intended to be limiting in any way.

The term “about” is used herein to mean approximately, in the region of, roughly, or around. When the term “about” is used in conjunction with a numerical range, it modifies that range by extending the boundaries above and below the numerical values set forth. The term “comprises” is used herein to mean “includes, but is not limited to.”

In a non-limiting embodiment of one aspect of the invention, a retrosynthesis of DHA shown by Scheme I is provided. Compounds represented by Formula I and Formula II are coupled in the presence of CuI, NaI and K₂CO₃ in DMF. The resulting compound represented by Formula III is partially hydrogenated by using Lindlar catalyst, and followed by hydrolysis of the ester of Formula XIV to yield DHA as the final product.

In a non-limiting embodiment of another aspect of the invention a method of preparing the compound represented by Formula I is provided. The synthetic route for this method is described in Scheme II and is conducted in the following two non-limiting steps:
(i) Preparation of a compound represented by Formula V: Compound represented by Formula IV is obtained by alkylation of propargyl alcohol with ethyl iodide by using n-BuLi, HMPA in THF at −78° C. to RT. The compound represented by Formula IV is taken forward in THF solvent to the tosylation reaction by using TsCl and KOH to obtain the compound represented by Formula V. The crude compound represented by Formula V is purified by silica gel column chromatography to finally obtain pure compound represented by Formula V with 33-40% yield.
(ii) Preparation of the compound represented by Formula I: The preparation of the compound represented by Formula I involves five steps including: DHP protection, Grignard reaction, coupling reaction, deprotection and tosylation. The DHP protection of propargyl alcohol gives a quantitative yield of the compound represented by Formula VI. The magnesium acetylide of the compound represented by Formula VI is reacted with propargyl bromide in presence of CuCl to obtain the compound represented by Formula VII with 65-75% yield. The coupling of the compound represented by Formula VII with the compound represented by Formula V in DMF provides the compound represented by Formula VIII. The crude compound represented by Formula VIII is subjected to deprotection in p-TSA/methanol at 60° C. for 3 hours to obtain the compound represented by Formula IX with 50% yield after silica gel column chromatography. Finally, the tosylation of the compound represented by Formula IX by using tosyl chloride in pyridine as solvent provides the compound represented by Formula I with 60-65% yield.

A method for the preparation of the compound represented by Formula V is also provided. The reaction scheme involved in a non-limiting embodiment of the method is as follows:

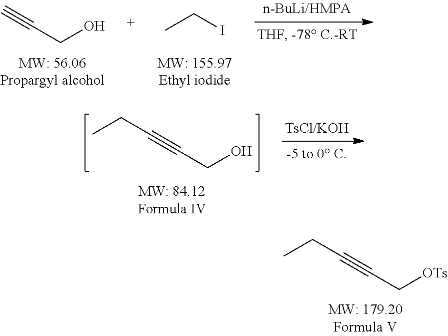

Without wishing to be limiting in any way, the raw materials that can be used for this method are illustrated in the Table 1 as follows:

TABLE 1

| | | | | | | | Remarks |
|---|---|---|---|---|---|---|---|
| S. No. | Name of the Material | Qty. | | M. Wt. | Moles | Mole Ratio | (Source/CAS etc.,) |
| 1. | Propargyl alcohol | 1 | Kg | 56 | 17.85 | 1.0 | |
| 2. | Ethyl iodide | 2.14 | L | 155.97 | 26.75 | 1.5 | d = 1.95 |
| 3. | n-butyl lithium, 2.5M in | 15 | L | 64 | 37.48 | 2.1 | Sainor laboratories |
| | n-hexane. | | | | | | |
| 4. | Hexamethylphosporamide | 9.3 | L | 179 | 53.55 | 3.0 | d = 1.03 |
| | (HMPA) | | | | | | |
| 5. | Tetrahydrofuran | 20 | L | — | — | 20 vol. | |
| 6. | Ice chilled 3N HCl (Lot-I) | 20 | L | 36.5 | 60 | 3.4 | |
| 7. | MTBE (Lot-I) | 10 | L | — | — | 10 vol. | |
| 8. | MTBE (Lot-II) | 10 | L | — | — | 10 vol. | |
| 9. | Ice chilled 1N HCl | 10 | L | | | | |
| 10. | Sat. NaCl solution | 10 | L | — | — | 10 vol. | |
| 11. | Anhydrous Na₂SO₄ | 1 | Kg | — | — | — | |
| 12. | Tosyl chloride | 3.4 | Kg | 190.65 | 17.85 | 1.0 | |
| 13. | KOH powder | 1.5 | Kg | 56 | 26.75 | 1.5 | |
| 14. | Ice chilled 3N Hydrochloric | 10 | L | 36.5 | 30 | 1.69 | |
| | acid (Lot-II) | | | | | | |
| 15. | MTBE (Lot-III) | 5 | L | — | — | — | |
| 16. | Sat. NaCl solution | 6 | L | — | — | 6 vol. | |

Example of the preparation of the compound represented by Formula V: In this exemplary, non-limiting method, THF is charged in to a clean and dry 20 L reactor, equipped with nitrogen inlet and a calcium chloride guard tube. Propargyl alcohol is charged in to the reactor until a clear solution in THF is obtained. HMPA (9.3 L) is added to the reaction mixture at room temperature (25-27° C.). Reaction mixture is cooled to −78° C. using dry ice/acetone bath. n-Butyl lithium (2.5M in hexane) is added to the reaction mixture over a period of 3 h, maintaining the temperature below −65° C. (−75 to −65° C.). The reaction mixture is maintained at −75° C. for 1 hr. Then, ethyl iodide is added slowly over a period of 1 hour maintaining the internal temperature within −75 to −65° C. until the pink colored reaction mixture changes to off-white color. Reaction mixture is allowed to RT (25 to 27° C.) and is stirred for 18 h.

The reaction progress is monitored by quenching small aliquots with water, extracting with ether, spotting over an analytical silica gel TLC plate (20% Ethyl acetate in hexane) and visualizing spots using KMnO₄ solution and anisaldehyde stains. After completion of the reaction, the reaction mixture is cooled to 0° C. using ice/water bath and then quenched with ice chilled 3N HCl (20 L) over a period of 1 hour. Organic layer (THF and Hexanes) is separated, and the aqueous layer is extracted with methyl tert-butyl ether (MTBE) (2×10 L; Lot I and II). The combined organic layer is washed with 1N HCl (1×10 L), followed by saturated sodium chloride solution (1×10 L). The organic phase is dried over anhydrous sodium sulphate and evaporated to a volume of 10 L under atmospheric pressure below 60° C. The THF layer, rich with the compound represented by Formula IV, is obtained as pale yellow solution, and is taken further for the tosylation reactions.

The THF layer, rich with the compound represented by Formula IV, is charged in to a clean and dry 20 L round bottomed flask (RBF) equipped with thermo pocket, mechanical stirrer and nitrogen atmosphere. Tosyl chloride (3.4 Kg) is added to reaction mixture at 25 to 30° C. The reaction mixture is cooled to −5 to 0° C. KOH (1.5 Kg) is added to reaction mixture in 20 portions over three hours maintaining the reaction mixture below 0° C. During the addition, the reaction temperature is maintained between −5° C. to +5° C. The reaction temperature (0-5° C.) is maintained for 1 hour. The reaction progress is monitored by quenching small aliquots of reaction mass with dilute HCl, extracting with ether, spotting over an analytical silica gel TLC plate (20% Ethyl acetate in hexanes) and, visualizing spots using anisaldehyde stain.

After the completion of reaction, the reaction mixture is quenched by slow addition of 3N HCl (Lot-II) within 0-10° C. The organic layer is separated and aqueous layer is extracted with MTBE (Lot-III). The combined organic layer is washed with saturated NaCl (2×3 L) and dried over anhydrous sodium sulphate. The organic layer is distilled under vacuum at 60° C. to obtain a brown colored oil which is the crude compound represented by Formula V. The crude is then purified by silica gel column chromatography to provide 1.35 Kg of pure compound represented by Formula V.

A method for the preparation of the compound represented by Formula VI is also provided. The reaction scheme involved in a non-limiting embodiment of the method is as follows:

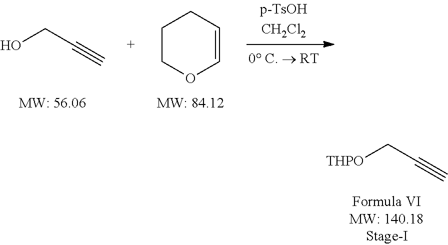

Without wishing to be limiting in any way, the raw materials that can be used for this method are illustrated in the Table 2 as follows:

TABLE 2

| S. | Name of the | | | | | Mole |
|---|---|---|---|---|---|---|
| No. | Material | Qty. | | M. Wt. | Moles | Ratio |
| 1. | Propargyl alcohol | 1 | Kg | 56 | 17.85 | 1.0 |
| 2. | 3,4-Dihydro-2H-pyran | 1.67 | L | 84.1 | 19.64 | 1.1 |
| 3. | p-Toluene sulfonic | 34.3 | g | 190.65 | 0.18 | 0.01 |
| | acid (PTSA) | | | | | |
| 4. | Dichloromethane | 8 | L | — | — | 8.0 vol. |
| | (Lot-I) | | | | | |
| 5. | Dichloromethane | 1 | L | — | — | 1.0 vol. |
| | (Lot-II) | | | | | |
| 6. | NaHCO₃ (s) | 336 | g | 84 | 4.0 | 0.22 |
| 7. | NaHCO₃ solution | 0.5 | L | | | 0.5 vol. |
| 8. | Sat. NaCl solution | 0.5 | L | | | 0.5 vol. |

Example of the preparation of the compound represented by Formula VI: In this exemplary, non-limiting method, dichloromethane (Lot-I) is charged in to a clean dry 20 L multi-neck round bottom flask equipped with mechanical stirrer. Propargyl alcohol (1 Kg) is charged in to the reactor. PTSA (34.4 g) is charged in to the reactor in one lot. The reaction mixture is chilled to 0° C. using an ice/water bath. 3,4-Dihydro-2H-pyran (1.67 L) is then diluted with dichloromethane (DCM) (Lot-II) and is then added to the reaction mixture over 1.5 hours maintaining the reaction temperature at 0-5° C. The reaction mixture is allowed to stir at 0-5° C. for 3 hours. The reaction progress is monitored by taking a small aliquot, and by spotting over an analytical silica gel TLC plate.

After the completion of reaction, the reaction mass is quenched (basified) by adding excess of solid NaHCO₃ and stirring for 30 min. Water (1 L, 1 vol.) is added in to reaction mass and stirred for 15 min. The layers are separated and organic layer is washed with 10% NaHCO₃ (500 mL, 0.5 vol.). The organic layer is finally washed with saturated NaCl solution (500 mL, 0.5 vol.) and dried over anhydrous sodium sulfate. The organic layer is evaporated under reduced pressure to obtain the crude compound represented by Formula VI.

A method for the preparation of the compound represented by Formula VII is also provided. The reaction scheme involved in a non-limiting embodiment of the method is as follows:

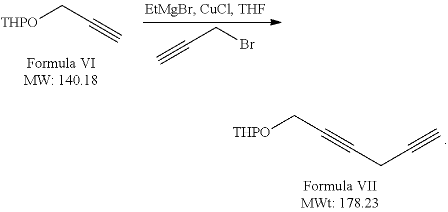

Without wishing to be limiting in any way, the raw materials that can be used for this method are illustrated in the Table 3 as follows:

TABLE 3

| S. | Name of the | | | | | Mole |
|---|---|---|---|---|---|---|
| No. | Material | Qty. | | M. Wt. | Moles | Ratio |
| 1. | Compound represented | 500 | g | 140.18 | 3.57 | 1.0 |
| | by Formula VI | | | | | |
| 2. | Propargyl bromide | 439 | mL | 119 | 3.92 | 1.1 |
| | (80 wt % in toluene, | | | | | |
| | d = 1.33) | | | | | |
| 3. | Ethyl magnesium | 3.92 | L | 133 | 3.92 | 1.1 |
| | bromide (1M in THF) | | | | | |
| 4. | CuCl | 3.53 | g | 99 | 0.036 | .01 |
| 5. | THF | 500 | mL | — | — | 1 vol. |
| 6. | MTBE | 3.0 | L | — | — | 6 vol. |
| 7. | Sat. NH₄Cl solution | 2.0 | L | — | — | 4 vol. |
| 8. | Sat. NaCl solution | 1.0 | L | — | — | 2 vol. |

Example of the preparation of the compound represented by Formula VII: In this exemplary, non-limiting method, ethyl magnesium bromide (3.92 L, 1M solution in THF) is charged in to a 20 L multi-neck round bottom flask, equipped with mechanical stirrer, argon inlet, reflux condenser and thermometer pocket. The compound represented by Formula VI (500 g) dissolved in THF is charged in to reaction mixture using addition funnel over 1 hour while maintaining the reaction temperature below 40° C.

The reaction mass is slowly warmed using water bath to maintain gentle reflux (65-70° C.) for 30 min. The reaction mass is then allowed to turn from black to brown after the addition of the compound represented by Formula VI. The water bath is replaced by ice bath and the reaction mixture is cooled to 10° C. Copper (I) chloride (3.53 g) is charged in to reaction mixture in one lot under argon atmosphere and the reaction mixture is stirred at 10° C. for 15 min. Propargyl bromide (439 mL) is then charged in to reaction mass using addition funnel over 30 min. The reaction mass is warmed to reflux temperature (65-70° C.) for 5 hours and heating is turned off at the end of 5 hours. The reaction mass is stirred at room temperature for 7 hours. The reaction progress is monitored by taking a small aliquot and by spotting over an analytical silica gel TLC plate (10% Ethyl acetate in hexanes and, visualizing spots using anisaldehyde solution).

The reaction mixture is diluted with MTBE. Saturated NH₄Cl solution is added and the reaction mass is stirred for 15 min. The two layers are separated. Organic layer is then washed with water (3×1 L), brine solution (1 L) and dried over anhydrous sodium sulfate. Organic layer is concentrated under reduced pressure to obtain 640 g of the crude compound represented by Formula VII. The crude is purified by silica gel column chromatography.

A method of preparing the compound represented by Formula VIII is also provided. The reaction scheme involved in a non-limiting embodiment of the method is as follows:

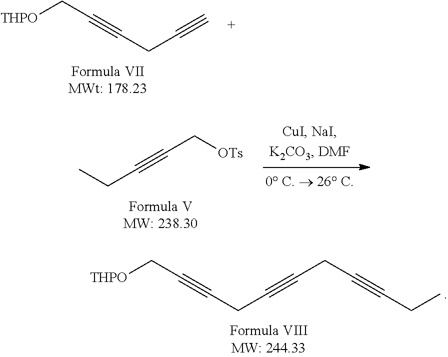

Without wishing to be limiting in any way, the raw materials that can be used for this method are illustrated in the Table 4 as follows:

TABLE 4

| S. | Name of the | | | | | Mole |
|---|---|---|---|---|---|---|
| No. | Material | Qty. | | M. Wt. | Moles | Ratio |
| 1. | Compound | 500 | g | 178.23 | 2.81 | 1 |
| | represented | | | | | |
| | by Formula VII | | | | | |
| 2. | Compound | 936.5 | g | 238.3 | 3.93 | 1.4 |
| | represented | | | | | |
| | by Formula V | | | | | |
| 3. | CuI | 374 | g | 190.45 | 1.96 | 0.7 |
| 4. | NaI | 589 | g | 149.9 | 3.93 | 1.4 |
| 5. | K₂CO₃ | 543 | g | 138.2 | 3.93 | 1.4 |
| 6. | Dimethyl | 3 | L | — | — | 6 vol. |
| | formamide | | | | | |
| | (Lot-I) | | | | | |
| 7. | Dimethyl | 1 | L | — | — | 2 vol. |
| | formamide | | | | | |
| | (Lot-II) | | | | | |
| 8. | MTBE (Lot-I) | 3 | L | — | — | 6 vol. |
| 9. | Water | 3 | L | — | — | 6 vol. |
| 10. | MTBE (Lot-II) | 3 | L | — | — | 6 vol. |
| 11. | MTBE (Lot-III) | 3 | L | — | — | 6 vol. |
| 12. | Sat. NaCl | 2 | L | — | — | 4 vol. |
| | solution | | | | | |
| 13. | Water | 3 × 3 | L | — | — | 3 × 6 vol. |

Example of the preparation of the compound represented by Formula VIII: In this exemplary, non-limiting method, DMF (3 L, 6 vol.) is charged in to a 20 L multi-neck flask equipped with mechanical stirrer, argon bubbler and 1 L addition funnel and the solvent is degassed for 15 min. CuI, sodium iodide and potassium iodide are then charged in to the reactor. The reaction mass is warmed to 30-35° C. until the reaction mass turns to yellow color. The reaction mass is chilled to 0° C. using ice/water bath. The compound represented by Formula VII is added to reaction mass over 5 min. The reaction mixture is stirred at 0-5° C. for 15 minutes maintaining the degassing conditions. Using the addition funnel, the compound represented by Formula V is added in to reaction mass over 30 minutes and the degassing is continued for another 30 min. The argon flow is slightly reduced so as to maintain inert atmosphere in the reaction flask and the stirring is continued for 18 hours. The reaction progress is monitored by taking a small aliquot, by quenching with water and MTBE, and by spotting organic layer over an analytical silica gel TLC plate.

To the reaction mass MTBE (Lot I), water is added and stirred for 15 minutes. The reaction mixture is filtered on Celite™ pad and washed with MTBE (Lot-II). The layers of filtrate are separated and the aqueous layer is extracted with MTBE (Lot-III). The combined organic layer is washed with water (3×3 L; until there are no solid precipitation observed) and with saturated NaCl (1×2 L). The organic layer is dried over anhydrous sodium sulfate and concentrated under reduced pressure to obtain the crude compound represented by Formula VIII.

A method of preparing the compound represented by Formula IX is also provided. The reaction scheme involved in a non-limiting embodiment of the method is as follows:

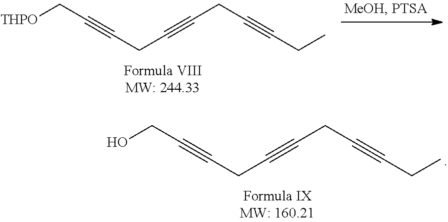

Without wishing to be limiting in any way, the raw materials that can be used for this method are illustrated in the Table 5 as follows:

TABLE 5

| | | | | | | | Remarks |
|---|---|---|---|---|---|---|---|
| S. No. | Name of the Material | Qty. | | M. Wt. | Moles | Mole Ratio | (Source/CAS etc.,) |
| 1 | Crude compound | 180 | g | 244.33 | 0.74 | 1.0 | 180 g of compound |
| | represented | | | | | | represented by Formula |
| | by Formula VIII | | | | | | VIII was obtained from |
| | | | | | | | 130 g of compound |
| | | | | | | | represented by Formula |
| | | | | | | | VII. |
| 2 | p-Toluene | 1.4 | g | 190.65 | 0.0074 | 0.01 | |
| | sulfonic acid | | | | | | |
| | (PTSA) | | | | | | |
| 3 | Methanol | 900 | mL | — | — | 5.0 vol. | |

Example of the preparation of the compound represented by Formula IX: In this exemplary, non-limiting method, methanol (900 mL) is taken into a 2 L multi-necked RBF equipped with magnetic stirrer, reflux condenser, thermo pocket, and heating provision. The compound represented by Formula VIII (180 g) and PTSA (1.4 g) are added to the reaction mixture at 25 to 30° C. The reaction temperature is raised slowly using an oil bath to maintain a gentle reflux (65-70° C.). Reflux conditions are maintained for 3 hours under inert atmosphere. The reaction progress is monitored by spotting over an analytical silica gel TLC plate, and by visualizing spots using KMnO₄ and anisaldehyde stains. The reaction mixture is cooled to room temperature and concentrated under vacuum at 60-70° C. The final product is purified by column chromatography using silica gel (60-120 mesh) to obtain 58 g of pure compound represented by Formula IX.

A method of preparing the compound represented by Formula I from the compound represented by Formula IX is also provided. The reaction scheme involved in a non-limiting embodiment of the method is as follows:

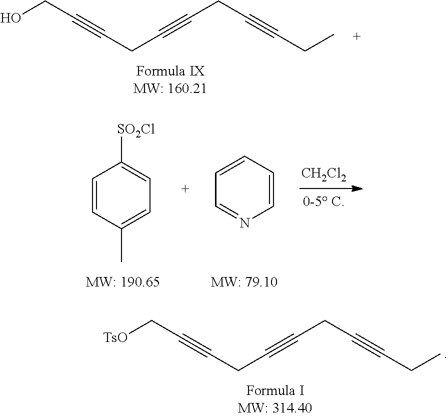

Without wishing to be limiting in any way, the raw materials that can be used for this method are illustrated in the Table 6 as follows:

TABLE 6

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | Compound represented by | 25 | g | 160.21 | 0.16 | 1 | | |
| | Formula IX | | | | | | | |
| 2. | TsCl | 59.5 | g | 190.65 | 0.31 | 2 | | |
| 3. | Pyridine | 75 | mL | 79.1 | — | 3 | vol. | d = 0.98 |
| 4. | Dichloromethane (Lot-I) | 25 | mL | — | — | 1 | vol. | |
| 5. | Dichloromethane (Lot-II) | 87.5 | mL | — | — | 3.5 | vol. | |
| 6. | MTBE (Lot-I) | 500 | mL | — | — | 20 | vol. | |
| 7. | MTBE (Lot-II) | 250 | mL | — | — | 10 | vol. | |
| 6. | 3N Hydrochloric acid | 350 | mL | — | — | 14 | vol. | |
| 7. | 1M Hydrochloric acid | 250 | mL | — | — | 2 × 5 | vol. | |
| 8. | Saturated NaCl solution | 250 | mL | — | — | 10 | vol. | |

Example of the preparation of the compound represented by Formula I: In this exemplary, non-limiting method, pyridine is charged in to a 1 L multi neck RBF, equipped with N₂ inlet, additional funnel and magnetic stirrer. Reaction mixture is cooled to −5 to 0° C. The compound represented by Formula IX in dichloromethane (Lot-I) is added slowly to the reaction mixture over a period of 15 minutes at −5 to 0° C. till a dark brown colored solution is observed. The reaction mixture is stirred at −5° C. for 10 minutes. Tosyl chloride in dichloromethane (Lot-II) is added to the reaction mixture over a period of 30 minutes at −5 to 0° C. The addition of tosyl chloride is controlled so as to maintain the reaction temperature below 5° C. Reaction mixture is stirred at the same temperature for 1 hour. Reaction is monitored by TLC analysis by spotting over an analytical silica gel TLC plate (20% Ethyl acetate in hexanes), and by visualizing spots using KMnO₄ and anisaldehyde stain.

Reaction mixture is quenched with 3N hydrochloric acid (350 mL). During the process of quenching brown insoluble particles are formed. The reaction mixture is diluted with MTBE (Lot-I) and stirred at 25 to 30° C. for 5 minutes. The two layers are separated and the aqueous layer is extracted with MTBE (Lot-II). The combined organic layer is washed with 1N Hydrochloric acid (2×5 vol.). The organic layer is washed with saturated NaCl solution (10 vol.), is dried over anhydrous sodium sulfate and evaporated under reduced pressure at 45° C. The final product, the compound represented by Formula I, is obtained as a dark brown liquid.

In a non-limiting embodiment of another aspect of the invention, there is provided a method of preparing the compound represented by Formula II. The synthetic route is described in Scheme III, and without wishing to be limiting in any way, is conducted in five stages including tosylation, coupling reaction, tosylation, coupling reaction and silyl deprotection.

Example of the preparation of the compound represented by Formula II: In this exemplary, non-limiting method, 2-Butyne-1,4-diol is tosylated with tosyl chloride and pyridine in dichloromethane to obtain the compound represented by Formula X with 45-50% yield. The crude compound represented by Formula X is reacted with trimethylsilyl acetylene to obtain the compound represented by Formula XI with 60-65% yield. Tosylation of the compound represented by Formula XI in pyridine provides the compound represented by Formula XII with 65-75% yield. The tosylated compound is then treated with methyl-4-pentynoate to provide the compound represented by Formula XIII with 45% yield. Deprotection of the compound represented by Formula XIII using TBAF/Dichloromethane gives the compound represented by Formula II with 65% yield.

A method of preparing the compound represented by Formula X is also provided. The reaction scheme involved in a non-limiting embodiment of the method is as follows:

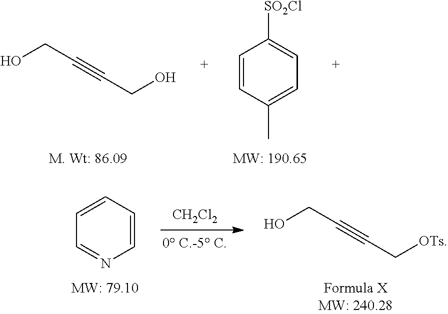

Without wishing to be limiting in any way, the raw materials that can be used for this method are illustrated in the Table 7 as follows:

TABLE 7

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | 2-Butyne-1,4-diol | 1 | Kg | 86 | 11.62 | 1 | | |
| 2. | P-TsCl | 1.995 | Kg | 190.65 | 10.46 | 0.9 | | |
| 3. | Pyridine | 1.873 | L | 79 | 23.24 | 2 | | d = 0.98 |
| 4. | Dichloromethane (Lot-I) | 25 | L | — | — | 25 | vol. | |
| 5. | Dichloromethane (Lot-II) | 15 | L | — | — | 15 | vol. | |
| 6. | 3N HCl | 8 | L | — | 25.56 | 2.2 | | |
| 7. | 1N HCl | 10 | L | — | — | 10 | vol. | |
| 8. | Methanol | 3 | L | — | — | 3 | vol. | |
| 9. | Sat. NaCl solution | 5 | L | — | — | 5 | vol. | |

Example of the preparation of the compound represented by Formula X: In this exemplary, non-limiting method, dichloromethane (25 L, lot-I) and 2-butynel,4-diol (1 Kg) are charged in to a reactor. Reaction mixture is cooled to 0° C. Pyridine is added to the reaction mixture over a period of 10 minutes at −5 to 0° C. Tosyl chloride in dichloromethane (Lot-II) is added to the reaction mixture over a period of 3 h at −5 to 0° C. Reaction mixture is stirred at the same temperature for 4 h then quenched with 3N hydrochloric acid. Quenching is done below 10° C. The two layers are separated and the organic layer is washed with 1N hydrochloric acid.

The combined organic layer is washed with brine solution (5 L). The organic layer is dried over anhydrous sodium sulfate and evaporated under reduced pressure at 45° C. The dark brown liquid obtained is taken in methanol (3 L). The reaction mixture is cooled to 0-5° C. until white solids crashed out from the methanolic solution. The suspended solids are filtered. The compound rich filtrate is evaporated under reduced pressure at 50° C. to obtain the compound represented by Formula X.

A method of preparing the compound represented by Formula XI is also provided. The reaction scheme involved in a non-limiting embodiment of the method is as follows:

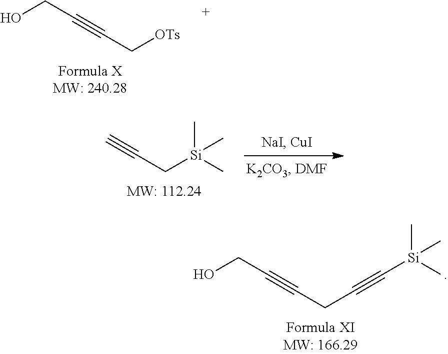

Without wishing to be limiting in any way, the raw materials that can be used for this method are illustrated in the Table 8 as follows:

TABLE 8

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | Compound represented | 450 | g | 240.28 | 1.87 | 1 | | |
| | by Formula X | | | | | | | |
| 2. | Trimethyl acetylene | 368 | g | 98.22 | 3.74 | 2 | | |
| 3. | CuI | 250 | g | 190.45 | 1.31 | 0.7 | | |
| 4. | NaI | 393 | g | 149.9 | 2.62 | 1.4 | | |
| 4. | K₂CO₃ | 362 | g | 138.2 | 2.62 | 1.4 | | |
| 5. | Dimethyl formamide | 3.15 | L | — | — | 7 | vol. | |
| | (Lot-I) | | | | | | | |
| 6. | Water (Lot-I) | 0.9 | L | — | — | 2 | vol. | |
| 7. | MTBE (Lot-I) | 1.8 | L | — | — | 4 | vol. | |
| 8. | MTBE (Lot-II) | 1.8 | L | — | — | 4 | vol. | |
| 9. | MTBE (Lot-III) | 1.8 | L | — | — | 4 | vol. | |
| 10. | Sat. NaCl | 1.8 | L | — | — | 4 | vol. | |
| 11. | Water | 3 × 1.8 | L | — | — | 3 × 4 | vol. | |

Example of the preparation of the compound represented by Formula XI: In this exemplary, non-limiting method, DMF (Lot-I) is charged in to a 20 L multi-neck flask equipped with mechanical stirrer, argon bubbler, 1 L addition funnel and the solvent is degassed for 15 min. CuI, sodium iodide and potassium iodide are charged in to the reactor. The reaction mass is chilled to 0° C. using ice/water bath. Trimethylsilyl acetylene is added to reaction mass over 30 min. The reaction mixture is stirred at 0-5° C. for 30 minutes maintaining the degassing conditions. Using addition funnel, the compound represented by Formula X is added in to reaction mass over 30 minutes and the degassing is continued for another 30 min. The argon flow is slightly reduced so as to maintain inert atmosphere in the reaction flask and the stirring is continued for 18 hours.

To the reaction mass, MTBE (Lot-I) and water (Lot-I) are added and stirred for 15 minutes. The reaction mixture is filtered on Celite™ pad and washed with MTBE (lot-II). The layers of filtrate are separated and the aqueous layer is extracted with MTBE (Lot-III). The combined organic layer is washed with water (3×1.8 L; until there are no solid precipitation observed) and then with saturated NaCl (1×1.8 L). The organic layer is dried over anhydrous sodium sulfate and concentrated on rotovap to obtain the crude compound represented by Formula XI-PHR-110. The crude is finally purified by silica gel column chromatography.

A method of preparing the compound represented by Formula XII is also provided. The reaction scheme involved in a non-limiting embodiment of the method is as follows:

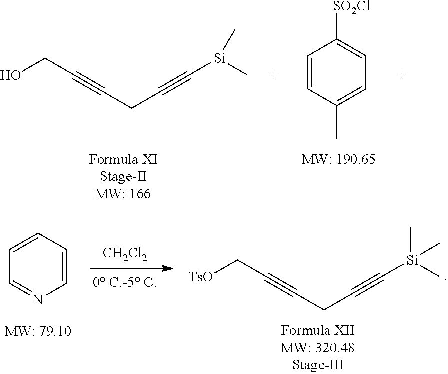

Without wishing to be limiting in any way, the raw materials that can be used for this method are illustrated in the Table 9 as follows:

TABLE 9

| S. | Name of the | | | | | Mole |
|---|---|---|---|---|---|---|
| No. | Material | Qty. | | M. Wt. | Moles | Ratio |
| 1 | Compound represented | 40 | g | 166 | 0.1807 | 1 |
| | by Formula XI | | | | | |
| 2 | Tosyl chloride | 92 | g | 190.65 | 0.3615 | 2 |
| 3 | pyridine | 120 | mL | — | — | 3 vol. |
| 4 | Dichloromethane | 40 | mL | — | — | 1 vol. |
| | (Lot-I) | | | | | |
| 5 | Dichloromethane | 140 | mL | — | — | 3.5 vol. |
| | (Lot-II) | | | | | |
| 6 | MTBE (Lot-I) | 360 | mL | — | — | 9 vol. |
| 7 | MTBE (Lot-II) | 180 | mL | — | — | 4.5 vol. |
| 8 | 3N Hydrochloric acid | 560 | mL | — | — | |
| 9 | 1N Hydrochloric acid | 480 | mL | — | — | 12 vol. |
| 10 | Saturated Sodium | 240 | mL | — | — | 6 vol. |
| | chloride | | | | | |

| | | | | | |
|---|---|---|---|---|---|
| 11 | Sodium sulfate | As | — | — | — |
| | anhydrous | needed | | | |

Example of the preparation of the compound represented by Formula XII: In this exemplary, non-limiting method, pyridine is charged in to a 1 L multi neck RBF equipped with a N₂ inlet, additional funnel and a magnetic stirrer. The reaction mixture is cooled to −5° C. The compound represented by Formula XI (40 g) in dichloromethane (Lot-I) is added slowly to the reaction mixture over a period of 15 minutes at −5 to 0° C. until a dark brown colored solution is observed. The reaction mixture is stirred at −5° C. for 10 minutes. Tosyl chloride dissolved in dichloromethane (Lot-II) is added to the reaction mixture over a period of 30 minutes at −5 to 0° C. The reaction mixture is then stirred at the same temperature for 1 h.

The reaction mixture is then quenched with 3N hydrochloric acid under 10° C. The reaction mixture is diluted with MTBE (Lot-I) and stirred at RT for 5 minutes until a dark brown, clear solution is observed. The two layers obtained are separated. The aqueous layer is again extracted with MTBE (Lot-II). The combined organic layer is washed with 1N hydrochloric acid (2×6 vol.) and the organic layer is washed with brine solution (6 vol.). The organic layer is dried over anhydrous sodium sulfate and evaporated under reduced pressure at 45° C. to a dark brown liquid which is the compound represented by Formula XII.

A method of preparing the compound represented by Formula XIII is also provided. The reaction scheme involved in a non-limiting embodiment of the method is as follows:

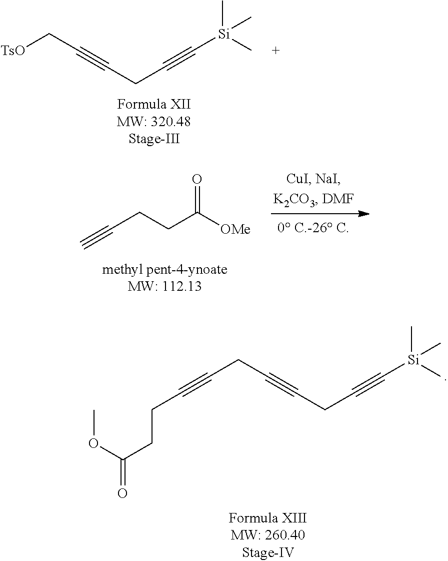

Without wishing to be limiting in any way, the raw materials that can be used for this method are illustrated in the Table 10 as follows:

TABLE 10

| S. | Name of the | | | | | Mole |
|---|---|---|---|---|---|---|
| No. | Material | Qty. | | M. Wt. | Moles | Ratio |
| 1. | Methyl-4-pentynoate | 20 | g | 112.13 | 0.178 | 1 |
| 2. | Compound represented | 80 | g | 320.38 | 0.249 | 1.4 |
| | by Formula XII | | | | | |
| 3. | CuI | 23.7 | g | 190.45 | 0.124 | 0.7 |
| 4. | NaI | 37.35 | g | 149.9 | 0.249 | 1.4 |
| 5. | K₂CO₃ | 34.44 | g | 138.2 | 0.249 | 1.4 |
| 6. | Dimethylformamide | 100 | mL | — | — | 5 vol. |
| | (Lot-I) | | | | | |
| 7. | Dimethylformamide | 20 | mL | — | — | 1 vol. |
| | (Lot-I) | | | | | |
| 8. | MTBE (Lot-I) | 500 | mL | — | — | 25 vol. |
| 9. | Water (Lot-I) | 20 | mL | — | — | 1 vol. |
| 10. | Water (Lot-II) | 200 | mL | — | — | 10 vol. |
| 11. | Water (Lot-III) | 200 | mL | — | — | 10 vol. |
| 12. | Sat. NaCl solution | 100 | mL | | | 5 vol. |

Example of the preparation of the compound represented by Formula XIII: In this exemplary, non-limiting method, DMF (100 mL, 5 vol.) is charged in to a 500 mL multi-neck flask equipped with mechanical stirrer, argon bubbler, and 250 mL addition funnel and the solvent is degassed for 15 min. CuI, sodium iodide and potassium iodide are charged in to the flask. The reaction mass is warmed to 30-35° C. and then it is chilled to 0° C. using ice/water bath. Methyl-4-pentynoate is added to the reaction mass over 5 min. The reaction mixture is stirred at 0-5° C. for 15 minutes maintaining the degassing conditions. Using addition funnel, the compound represented by Formula XII is added to the reaction mass over 10 minutes and the degassing is continued for another 30 min. The argon flow is slightly reduced so as to maintain inert atmosphere in the reaction flask and the stirring is continued for 18 hours.

To the reaction mass, MTBE (Lot I) and water (Lot I) are added and stirred for 15 minutes. The reaction mixture is filtered on Celite™ bed and washed with MTBE (Lot-II). The layers of filtrate are separated and the organic layer is washed with water (Lot-II and III). The organic layer is finally washed with brine and dried over anhydrous sodium sulfate. The organic layer is concentrated under reduced pressure to obtain the crude compound represented by Formula XIII.

A method of preparing the compound represented by Formula II is also provided. The reaction scheme involved in a non-limiting embodiment of the method is as follows:

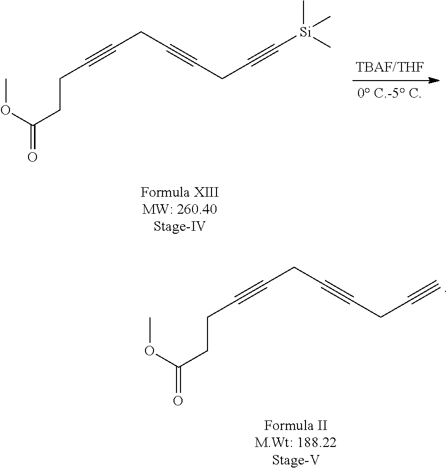 [SMILES: COC(=O)CCC#CCC#CCC#C[Si](C)(C)C ; C#CCC#CCC#CCCC(=O)OC]

Without wishing to be limiting in any way, the raw materials that can be used for this method are illustrated in the Table 11 as follows:

TABLE 11

| S. | Name of the | | | | | Mole |
|---|---|---|---|---|---|---|
| No. | Material | Qty. | | M. Wt. | Moles | Ratio |
| 1. | Compound represented | 16 | g | 260.4 | 0.0615 | 1 |
| | by Formula XIII | | | | | |
| 2. | TBAF | 9.69 | g | 315.5 | 0.0307 | 0.5 |
| 3. | Dichloromethane | 480 | mL | — | — | 30 vol. |
| 4. | Ice-Chilled water | 320 | mL | — | — | 20 vol. |
| 5. | Water | 320 | mL | — | — | 20 vol. |
| 6. | Sat. NaCl solution | 320 | mL | — | — | 20 vol. |

Example of the preparation of the compound represented by Formula II: In this exemplary, non-limiting method, dichloromethane (480 mL) and compound represented by Formula XIII (16 g) are charged in to a 1 L round bottom flask. The reaction mixture is cooled using salt ice/water bath to below 0° C. (−5 to 0° C.). Then, TBAF (9.69 g) is added to the reaction mixture over a period of 30 minutes in 10 lots maintaining the reaction temperature close to 0° C. Reaction mixture is stirred for 1 h at −5 to 0° C. and then it is quenched with ice-chilled water. The two layers obtained are separated. The organic layer is washed with water and then with saturated sodium chloride. The organic layer is dried over sodium sulfate and evaporated under reduced pressure to obtain the compound represented by Formula II as a black liquid. The crude is finally purified by silica gel column chromatography.

In a non-limiting embodiment of another aspect of the invention, there is provided a method of preparing DHA using the compounds represented by Formula I and II. The synthetic route is described in Scheme IV, and without wishing to be limiting in any way, the raw materials used for this method are illustrated in the Table 12 as follows:

TABLE 12

| S. | Name of the | | | | | Mole |
|---|---|---|---|---|---|---|
| No. | Material | Qty. | | M. Wt. | Moles | Ratio |
| 1. | Compound represented | 10 | g | 188.22 | 0.053 | 1 |
| | by Formula II | | | | | |
| 2. | Compound represented | 23.4 | g | 314.40 | 0.075 | 1.4 |
| | by Formula I | | | | | |
| 3. | CuI | 7.1 | g | 190.45 | 0.037 | 0.7 |
| 4. | NaI | 11.2 | g | 149.9 | 0.075 | 1.4 |
| 5. | K₂CO₃ | 10.3 | g | 138.2 | 0.075 | 1.4 |
| 6. | Dimethylformamide | 60 | mL | — | — | 6 vol. |
| | (Lot-I) | | | | | |
| 7. | Dimethylformamide | 60 | mL | — | — | 6 vol. |
| | (Lot-I) | | | | | |
| 8. | MTBE (Lot-I) | 250 | mL | — | — | 25 vol. |
| 9. | MTBE (Lot-II) | 250 | mL | — | — | 25 vol. |
| 10. | MTBE (Lot-III) | 250 | mL | — | — | 25 vol. |
| 11. | Water (Lot-I) | 10 | mL | — | — | 1 vol. |
| 12. | Water (Lot-II) | 100 | mL | — | — | 10 vol. |
| 13. | Water | 100 | mL | — | — | 10 vol. |
| 14. | Sat. NaCl solution | 50 | mL | | | 5 vol. |

Example of the preparation of DHA: In this exemplary, non-limiting method, the compounds represented by Formulas I and II are coupled in the presence of CuI, NaI and K2CO₃ in DMF to provide the compound represented by Formula III with 30-40% yield. A partial hydrogenation of the compound represented by Formula III using Lindlar's catalyst provides the ester represented by Formula XIV. Hydrolysis of the ester represented by the Formula XIV provides DHA.

The present invention will be further illustrated in the following examples.

### EXAMPLES

### Preparation of (4Z, 7Z, 10Z, 13Z, 16Z, 19Z)-docosa-4, 7, 10, 13, 16, 19-hexaenoic acid (DHA)

### Summary:

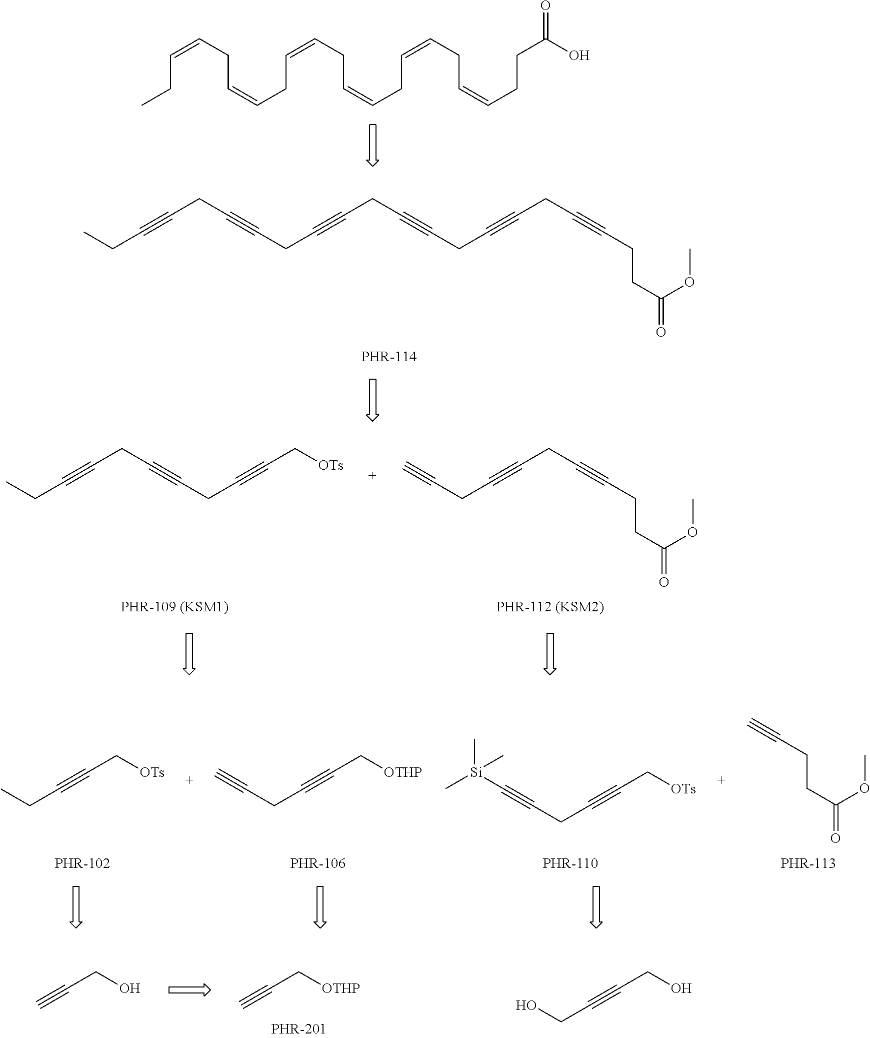

The key starting materials PHR-109 (KSM1) and PHR-112 (KSM2), both were prepared in the lab on multi gram scale from propargyl alcohol and 2-Butyne-1,4-diol respectively. The coupling reaction of both the KSM1 and 2 in the presence of CuI, NaI and K₂CO₃ in DMF gave PHR-114 in 30-40% yield. Partial hydrogenation of PHR-114 by using Lindlar's catalyst followed by hydrolysis of ester completed the synthesis of DHA.

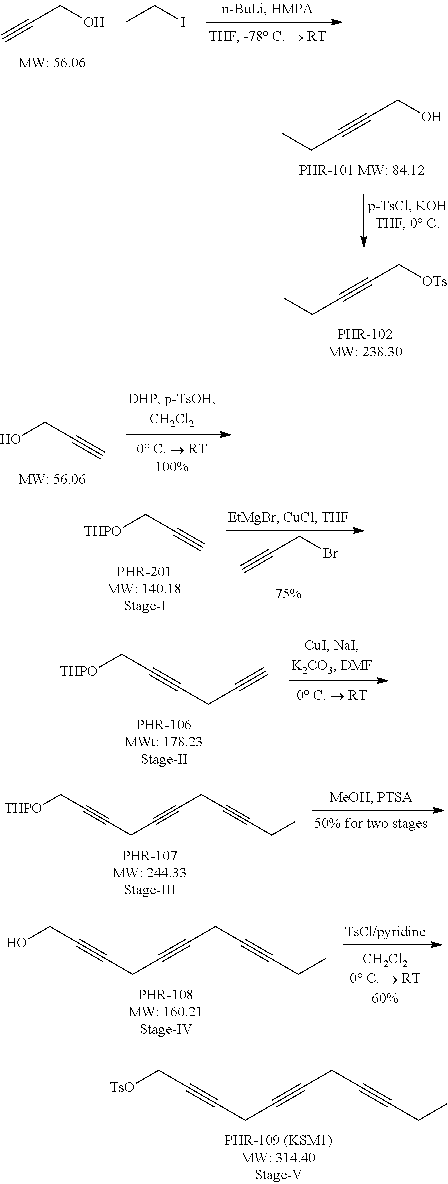

Preparation of PHR-102: Alkylation of propargyl alcohol with ethyl iodide by using n-BuLi, HMPA in THF at −78° C. to RT afforded PHR-101. The resulting PHR-101 in THF solvent was taken forward to the tosylation reaction by using TsCl and KOH to obtain PHR-102. The crude PHR-102 was purified by silica gel column chromatography to afford pure PHR-102 in 33-40% yield.

Preparation of PHR-109: The preparation of PHR-109 involved five steps i.e., DHP protection, Grignard reaction, coupling reaction, deprotection and tosylation. The DHP protection of propargyl alcohol gave quantitative yield of PHR-201. The magnesium acetylide of PHR-201 reacted with propargyl bromide in presence of CuCl to give PHR-106 in 65-75% yield. The coupling of PHR-106 with PHR-102 in DMF afforded PHR-107 and the crude was subjected to deprotection in p-TSA/methanol at 60° C. for 3 h to give PHR-108 in 50% yield after silica gel column chromatography. Tosylation of PHR-108 by using tosyl chloride in pyridine resulted PHR-109 in 60-65% yield.

The overall yield for the preparation of PHR-109 from propargyl alcohol was about 20%.

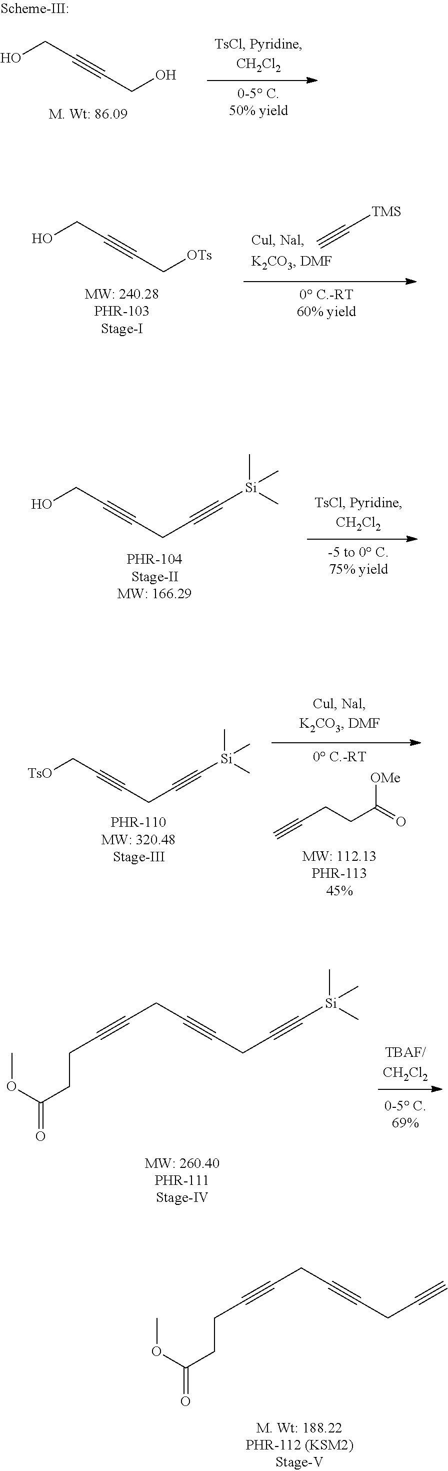

The preparation of PHR-112 involved five stages namely, tosylation, coupling reaction, tosylation, coupling reaction and silyl deprotection. The tosylation of 2-Butyne-1,4-diol with tosyl chloride, pyridine in dichloromethane gave PHR-103 in 45-50% yield. The crude PHR-103 was subjected to react with trimethylsilyl acetylene to give PHR-104 in 60-65% yield. The tosylation of PHR-104 in pyridine afforded PHR-110 in 65-75% yield. The tosylated compound was then treated with methyl-4-pentynoate to afford PHR-111 in 45% yield. Deprotection of PHR-111 using TBAF/Dichloromethane gave PHR-112 in 65% yield.

The overall yield for the preparation of PHR-112 from 2-Butyne-1,4-diol was about 6-7%.

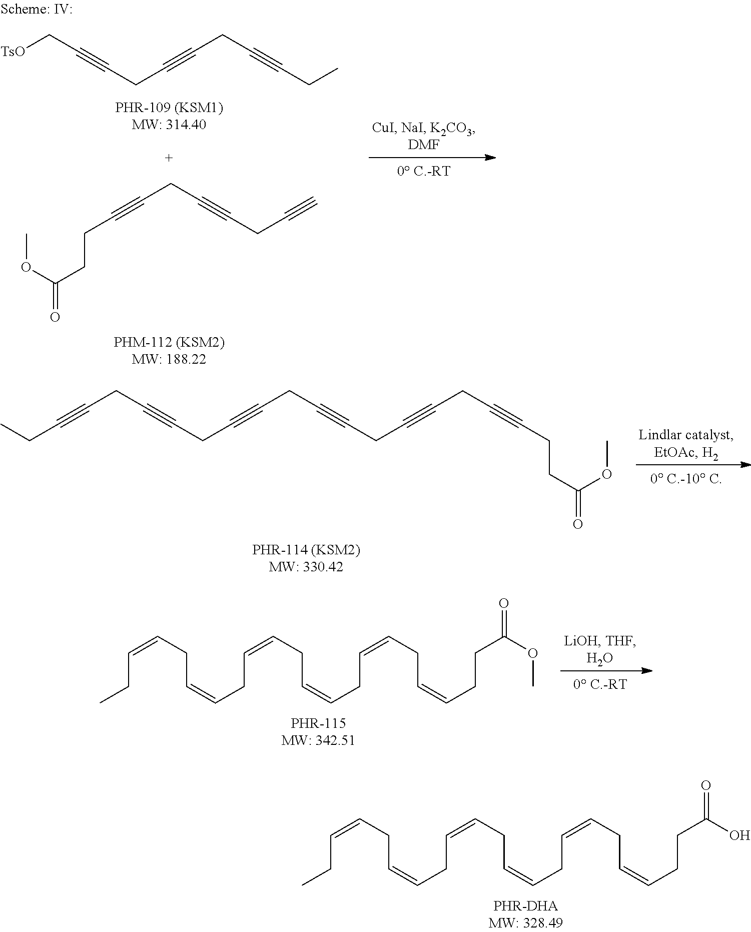

The coupling reaction of both the KSM1 and KSM2 in presence of CuI, NaI and K₂CO₃ in DMF gave PHR-114 in 30-40% yield. Partial hydrogenation of PHR-114 using Lindlar's catalyst followed by hydrolysis of ester completed the synthesis of DHA.

### Optimized Final Procedures:

### PHR-109 (KSM1): Preparation of PHR-102:

### Stage-I: Preparation of pent-2-yn-1-ol

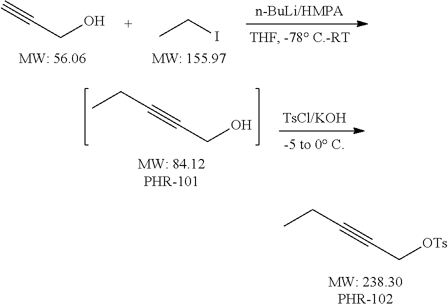

### Batch No #PAA-P-10-040-PHR-101-03

### Raw Materials:

| | | | | | | | Remarks |
|---|---|---|---|---|---|---|---|
| S. No. | Name of the Material | Qty. | | M. Wt. | Moles | Mole Ratio | (Source/CAS etc.,) |
| 1. | Propargyl alcohol | 1 | Kg | 56 | 17.85 | 1.0 | |
| 2. | Ethyl iodide | 2.14 | L | 155.97 | 26.75 | 1.5 | d = 1.95 |
| 3. | n-butyl lithium, 2.5M in | 15 | L | 64 | 37.48 | 2.1 | Sainor |
| | n-hexane. | | | | | | laboratories |
| 4. | Hexamethylphosporamide | 9.3 | L | 179 | 53.55 | 3.0 | d = 1.03 |
| | (HMPA) | | | | | | |
| 5. | Tetrahydrofuran | 20 | L | — | — | 20 vol. | commercial |
| 6. | Ice cold 3N HCl (Lot-I) | 20 | L | 36.5 | 60 | 3.4 | |
| 7. | MTBE (Lot-I) | 10 | L | — | — | 10 vol. | commercial |
| 8. | MTBE (Lot-II) | 10 | L | — | — | 10 vol. | commercial |
| 9. | Ice cold 1N HCl | 10 | L | | | | |
| 10. | Sat. NaCl solution | 10 | L | — | — | 10 vol. | |
| 11. | Anhydrous Na₂SO₄ | 1 | Kg | — | — | — | |
| 12. | Tosyl chloride | 3.4 | Kg | 190.65 | 17.85 | 1.0 | |
| 13. | KOH powder | 1.5 | Kg | 56 | 26.75 | 1.5 | |
| 14. | Ice cold 3N Hydrochloric | 10 | L | 36.5 | 30 | 1.69 | |
| | acid (Lot-II) | | | | | | |
| 15. | MTBE (Lot-III) | 5 | L | — | — | — | commercial |
| 16. | Sat. NaCl solution | 6 | L | — | — | 6 vol. | |

### Process Details:

### Step-I:

1. THF was charged into a clean and dry 100 L reactor.
2. Proargyl alcohol (1 Kg) was charged in to the reactor.
Observation: Clear solution in THF.
3. HMPA (9.3 L) was added to the reaction mixture at room temperature (25-30° C.) for 10 minutes.
4. Reaction mixture was cooled to −78° C. using dry ice/acetone bath.
5. n-Butyl lithium (2.5M in hexane) was added to the reaction mixture over a period of 3 h, maintaining the temperature below −65° C. (−75 to −65° C.).
Note: The reaction should be under nitrogen atmosphere.
Observation: The turbid and thick suspension was observed at the end of n-Butyl lithium addition and pink to red color was identified.
6. The reaction mixture was maintained at −75° C. for 1 h.
7. After the maintenance, ethyl iodide was added slowly over a period of 1 h maintaining the internal temperature within −75 to −65° C.
Observation: During addition, the pink color reaction mixture was changed to off white color.
8. Reaction mixture was allowed to RT (25 to 30° C.) and stirred for 18 h.
Note: The reaction progress was monitored by TLC analysis (20% Ethyl acetate in hexane) and visualizing spots using KMnO₄ solution and anisaldehyde stains. R_{f}=0.2 (PHR-101).
9. After completion of the reaction, the reaction mixture was cooled to 0° C. using ice/water bath and quenched with ice cold 3N HCl (20 L) over a period of 1 h.
10. Organic layer (THF and Hexanes) was separated and the aqueous layer was extracted with MTBE (2×10 L; Lot I and II).
11. The combined organic layer was washed with 1N HCl (1×10 L), followed by saturated sodium chloride solution (1×10 L).
12. The organic phase was dried over anhydrous sodium sulphate and evaporated to a volume of 10 L under atmospheric pressure below 60° C. 13. The PHR-101 rich THF layer was obtained as pale yellow solution which was taken further for the tosylation reactions.

| | |
|---|---|
| Weight of the product | Not isolated (compound in THF solvent). |
| Description | Yellow solution of THF |
| ¹H NMR | Complies with the structure |

¹H NMR data: (CDCl₃, 500 MHz, ppm): 4.22 (s, —OCH₂—, 2H), 2.21 (q, —CH₂CH₃, 2H), 1.85 (t, —CH₂CH₃, 3H).

### Step-II

14. PHR-101 rich THF layer was charged in to a clean and dry 50 L reactor.
15. Tosyl chloride (3.4 Kg) was added to reaction mixture at 25 to 30° C. for 10 minutes.
16. Cool the reaction mixture to −5 to 0° C.
17. KOH (1.5 Kg) was added to reaction mixture in 20 portions over 3 h maintaining the reaction mixture −5 to 0° C.
Note: During the addition, the reaction temperature should be maintained between −5° C. to 0° C.
18. Maintain the reaction temperature (−5 to 0° C.) for 1 h.
Note: The reaction progress was monitored by TLC analysis (20% Ethyl acetate in hexanes) and visualizing spots using anisaldehyde stain. R_{f}=0.2 (PHR-101), 0.3(PHR-102).
19. After the completion of reaction, the reaction mixture was quenched by slow addition of 3N HCl (Lot-II) within 0-10° C.
20. The organic layer was separated and aqueous layer was extracted with MTBE (Lot-III).
21. The combined organic layer was washed with saturated NaCl solution (2×3 L) and dried over anhydrous sodium sulphate.
22. The organic layer was distilled under vacuum at 60° C.
Observation: The crude PHR-102 was brown colored oil.
23. The crude was purified by silica gel column chromatography using 60-120 mesh silica and the compound eluted in 50% ethyl acetate in hexanes.

### Results

Weight of the product: 1.35 Kg
Yield: 33%
Description: Brown oil
¹H NMR: complies with the structure.

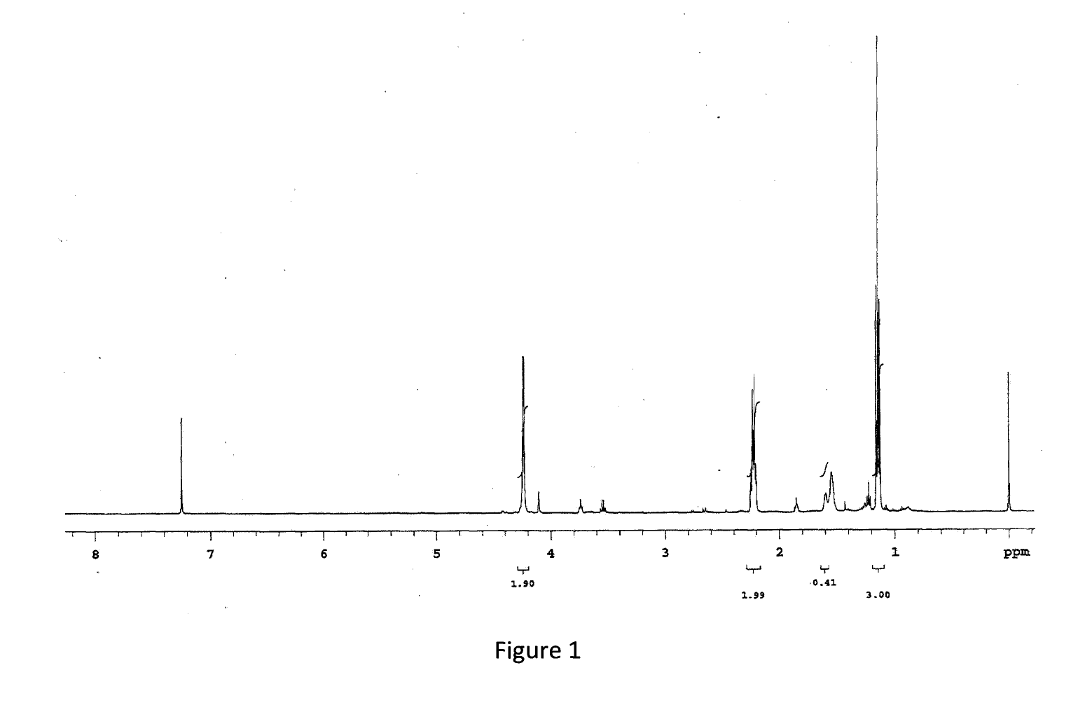

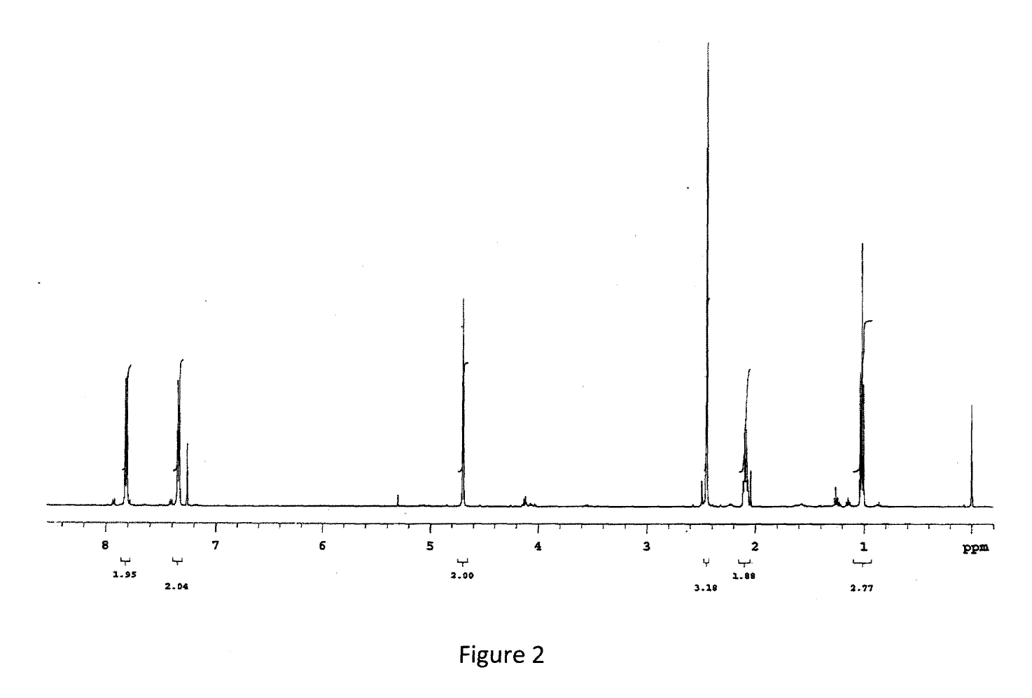

¹H NMR data: (CDCl₃, 500 MHz, ppm): 7.81 (d, ArCH, 2H), 7.38 (d, ArCH, 2H), 4.71 (s, —OCH₂—, 2H), 2.45 (s, ArCH₃, 3H), 2.08 (q, —CH₂CH₃, 2H), 1.02 (t, —CH₂CH₃, 3H) (FIGS. 1 and 2).

### Stage-I: Preparation of 2-(prop-2-ynyloxy)tetrahydro-2H-pyran

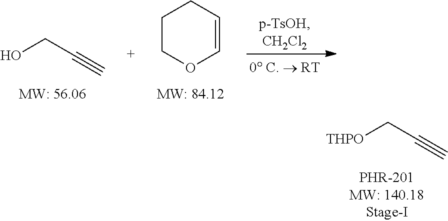

### Batch No #PAA-P-10-027-PHR-201-32

### Raw Materials:

| | | | | | | | Remarks |
|---|---|---|---|---|---|---|---|
| S. No. | Name of the Material | Qty. | | M. Wt. | Moles | Mole Ratio | (Source/CAS etc.,) |
| 1. | Propargyl alcohol | 1 | Kg | 56 | 17.85 | 1.0 | AVRA |
| | | | | | | | synthesis |
| 2. | 3,4-Dihydro-2H-pyran | 1.67 | L | 84.1 | 19.64 | 1.1 | |
| 3. | p-Toluene sulfonic | 34.3 | g | 190.65 | 0.18 | 0.01 | Commercial |
| | acid | | | | | | |
| 4. | Dichloromethane (Lot-I) | 8 | L | — | — | 8.0 vol. | Commercial |
| 5. | Dichloromethane (Lot-II) | 1 | L | — | — | 1.0 vol. | Commercial |
| 6. | NaHCO₃ (s) | 336 | g | 84 | 4.0 | 0.22 | Commercial |
| 7. | NaHCO₃ solution | 0.5 | L | | | 0.5 vol. | |
| 8. | Sat. NaCl solution | 0.5 | L | | | 0.5 vol. | |

### Process Details:

1. Dichloromethane (Lot-I) was charged in to a clean and dry 20 L 4 neck round bottom flask equipped with mechanical stirrer.
2. Propargyl alcohol (1 Kg) was charged in to the reactor.
3. PTSA (34.4 g) was charged in to the reactor in one lot.
4. The reaction mixture was cooled to −5 to 0° C.
5. 3,4-Dihydro-2H-pyran (1.67 L) was diluted with DCM (Lot-II) and then added to the reaction mixture over 1.5 h maintaining the reaction temperature at 0-5° C.
6. The reaction mixture was allowed to stir at 0-5° C. for 3 h.
Note: The reaction progress was monitored by TLC analysis (10% Ethyl acetate in hexanes and visualizing spots using anisaldehyde solution). R_{f}=0.6 (PHR-201) and 0.2 (Propargyl alcohol).
7. After completion of reaction, the reaction mixture was quenched (basified) by adding excess of solid NaHCO₃ and stirred for 30 min.
8. Water (1 L, 1 vol.) was added in to reaction mass and stirred for 15 min.
9. The layers were separated and organic layer was washed with 10% NaHCO₃ (500 mL, 0.5 vol.).
10. The organic layer was finally washed with saturated NaCl solution (500 mL, 0.5 vol.) and dried over anhydrous sodium sulfate
11. The organic layer was evaporated under reduced pressure to obtain crude PHR-201.

### Results

Weight of the product: 2.5 Kg
Yield: 100%
Description: Brown to black liquid
¹H NMR: complies with the structure.

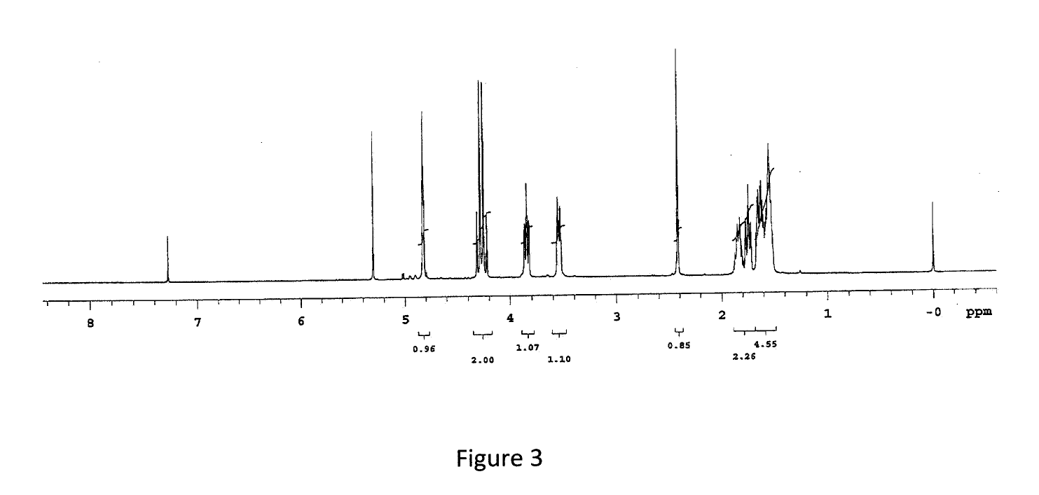

¹H NMR data: (CDCl₃, 500MHz, ppm): 4.81(t, —OCHO—,1H), 4.35-4.0 (dd, —OCH₂—, 2H), 3.9-3.8 (m, —OCH_{A}CH_{B}CH₂—, 1H), 3.57-3.5 (m, —OCH_{A}CH_{B}CH₂—, 1H), 2.40 (s, —CH, 1H), 1.9-1.7 (m, —CH₂—, 2H), 1.7-1.5 (m, 2×—CH₂—, 4H) (FIG. 3).

### Stage-II: Preparation of 2-(hexa-2,5-diynyloxy)tetrahydro-2H-pyran

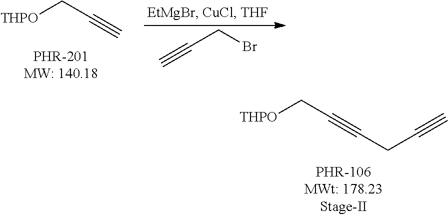

### Batch No #PAA-P-10-027-PHR-106-31

### Raw Materials:

| | | | | | | | Remarks |
|---|---|---|---|---|---|---|---|
| S. No. | Name of the Material | Qty. | | M. Wt. | Moles | Mole Ratio | (Source/CAS etc.,) |
| 1. | PHR-201 | 500 | g | 140.18 | 3.57 | 1.0 | |
| 2. | Propargyl bromide | 439 | mL | 119 | 3.92 | 1.1 | |
| | (80 wt % in toluene, d = 1.33) | | | | | | |
| 3. | EtMgBr (1M in THF) | 3.92 | L | 133 | 3.92 | 1.1 | Sainor Laboratories |
| 4. | CuCl | 3.53 | g | 99 | 0.036 | .01 | |
| 5. | THF | 500 | mL | — | — | 1 vol. | Commercial |
| 6. | MTBE | 3.0 | L | — | — | 6 vol. | Commercial |
| 7. | Sat. NH₄Cl solution | 2.0 | L | — | — | 4 vol. | |
| 8. | Sat. NaCl solution | 1.0 | L | — | — | 2 vol. | |

### Process Details:

1. Ethyl magnesium bromide (3.92 L, 1M solution in THF), was charged into a 20 L multi-neck round bottom flask, equipped with mechanical stirrer, argon inlet, reflux condenser and thermometer pocket.
2. PHR-201 (500 g) in THF was added to reaction mixture over 1 h maintaining the reaction temperature below 40° C.
3. The reaction mixture was slowly warmed using water bath to maintain gentle reflux (65-70° C.) for 30 min.
Note: The reaction mixture from black turned to brown color after addition of PHR-201.
4. The reaction mixture was cooled to 10° C.
5. Copper (I) chloride (3.53 g) was charged into reaction mixture in one lot under argon atmosphere and the reaction mixture was stirred at 10° C. for 15 min.
Note: The reaction mixture turned into grey color after addition of CuCl.
6. Propargyl bromide (439 mL) was added to reaction mixture over 30 min.
7. The reaction mixture was refluxed (65-70° C.) for 5 h.
Note: The reaction mixture became viscous and changed color to yellow and then to orange. In long time the reaction mass became brown color.
8. The reaction mixture was stirred at room temperature for 7 hours.
Note: The reaction progress was monitored by TLC analysis (10% Ethyl acetate in hexanes and, visualizing spots using anisaldehyde solution). R_{f}=0.5 (PHR-106) and 0.6 (PHR-201).
9. The reaction mixture was diluted with MTBE.
10. Saturated NH₄Cl solution was added and the reaction mass was stirred for 15 min.
11. The two layers were separated.
Observation: The aqueous layer was blue in color.
12. Organic layer was then washed with water (3×1 L), brine solution (1 L) and dried over anhydrous sodium sulfate.
13. Organic layer was concentrated under reduced pressure to obtain crude PHR-106.
Observation: 640 g of crude was obtained.
14. The crude was purified by silica gel column chromatography.

### Results

Weight of the product: 448 g
Yield: 70%
Description: Brown to black liquid
¹H NMR: complies with the structure

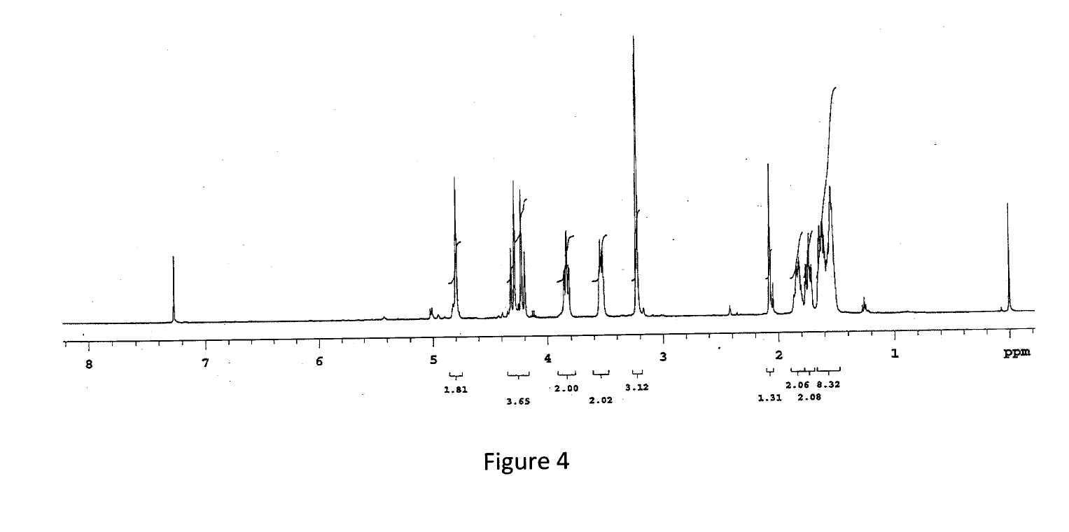

¹H NMR data: (CDCl₃, 500 MHz, ppm): 4.80 (t, —OCHO—, 1H), 4.35-4.19 (dd, —OCH₂—, 2H), 3.85-3.8 (m, —OCH_{A}CH_{B}CH₂—, 1H), 3.59-3.5 (m, —OCH_{A}CH_{B}CH₂—, 1H), 3.21 (s, —CH₂—, 2H), 2.08 (s, —CH, 1H), 1.9-1.65 (m, —CH₂—, 2H), 1.65-1.35 (m, 2×—CH₂—, 4H) (FIG. 4).

### Stage-III: Preparation of 2-(undeca-2,5,8-triynyloxy)tetrahydro-2H-pyran

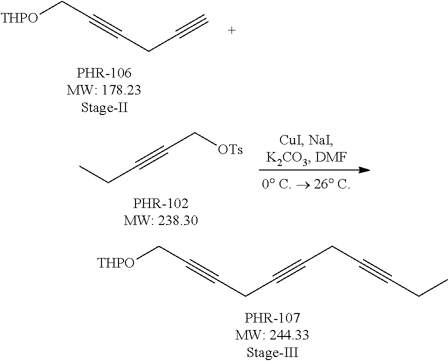

### Batch No #PAA-P-10-040-PHR-107-10

### Raw Materials:

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | PHR-106 | 500 | g | 178.23 | 2.81 | 1 | | |
| 2. | PHR-102 | 936.5 | g | 238.3 | 3.93 | 1.4 | | |
| 3. | CuI | 374 | g | 190.45 | 1.96 | 0.7 | | |
| 4. | NaI | 589 | g | 149.9 | 3.93 | 1.4 | | |
| 5. | K₂CO₃ | 543 | g | 138.2 | 3.93 | 1.4 | | |
| 6. | Dimethyl formamide | 3 | L | — | — | 6 | vol. | Commercial |
| | (Lot-I) | | | | | | | |
| 7. | Dimethyl formamide | 1 | L | — | — | 2 | vol. | Commercial |
| | (Lot-II) | | | | | | | |
| 8. | MTBE (Lot-I) | 3 | L | — | — | 6 | vol. | Commercial |
| 9. | Water | 3 | L | — | — | 6 | vol. | |
| 10. | MTBE (Lot-II) | 3 | L | — | — | 6 | vol. | Commercial |
| 11. | MTBE (Lot-III) | 3 | L | — | — | 6 | vol. | Commercial |
| 12. | Sat. NaCl solution | 2 | L | — | — | 4 | vol. | |
| 13. | Water | 3 × 3 | L | — | — | 3 × 6 | vol. | |

### Process Detail:

1. DMF (3 L, 6 vol.) was charged in to a 20 L multi-neck flask equipped with mechanical stirrer, argon bubbler and 1 L addition funnel and the solvent was degassed for 15 min.
2. CuI, Sodium iodide and potassium iodide were charged in to the reactor.
Note: The reaction mass turned in to yellow color with generation of heat (reaction mass was warmed to 30-35° C.
3. The reaction mixture was cooled to 0° C.
4. PHR-106 was added to reaction mixture over 5 min.
5. The reaction mixture was stirred at 0-5° C. for 15 minutes maintaining the degassing conditions.
6. PHR-102 was added in to reaction mixture over 30 minutes and the degassing was continued for another 30 minutes.
7. The argon flow was slightly reduced so as to maintain inert atmosphere in the reaction flask and the stirring was continued for 18 hours.
Note: The reaction progress was monitored by TLC analysis (5% Ethyl acetate in hexanes (double run) and visualizing spots using anisaldehyde solution). R_{f}=0.45 (PHR-106) and 0.5 (PHR-107).
8. MTBE (Lot I) and water was added to the reaction mixture and stirred for 15 minutes.
9. The reaction mixture was filtered on Celite™ pad and washed with MTBE (Lot-II)
10. The layers of filtrate were separated and the aqueous layer was extracted with MTBE (Lot-III).
11. The combined organic layer was washed with water (3×3 L; till there are no solid precipitation observed) and with saturated NaCl (1×2 L).
12. The organic layer was dried over anhydrous sodium sulfate and concentrated under reduced pressure to obtain the crude PHR-107.

### Results

Weight of the product: 580 g of crude
Yield: 85%
Description: Brown to black liquid
¹H NMR: complies with the structure

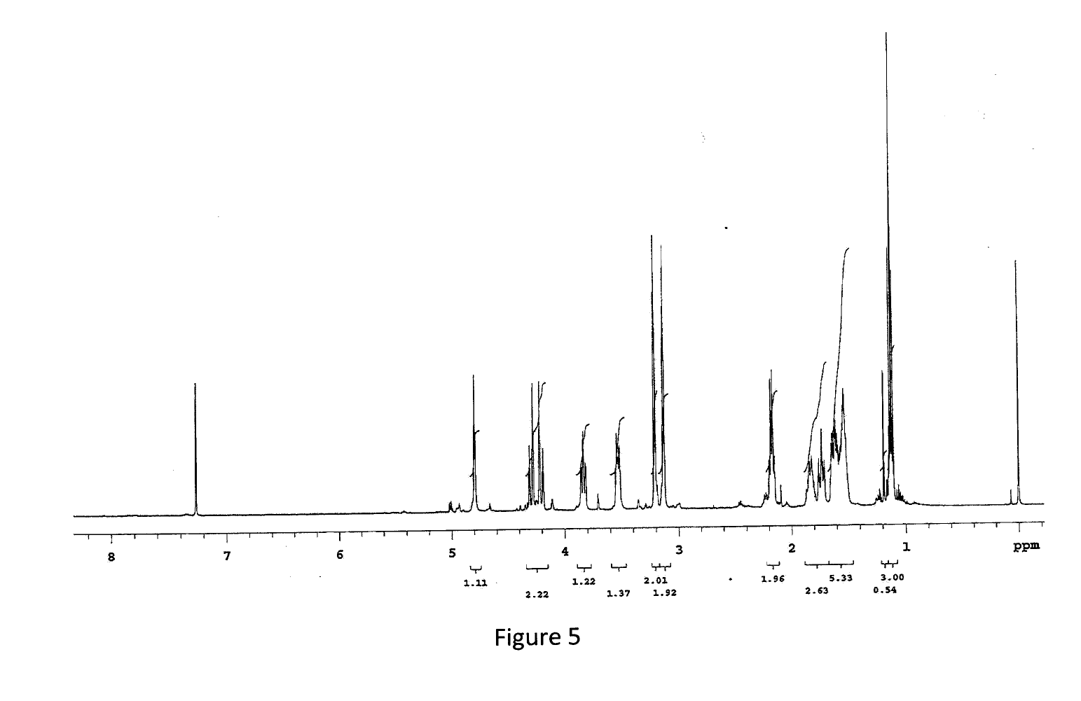

¹H NMR data: (CDCl₃, 500 MHz, ppm): 4.80 (t, —OCHO—, 1H), 4.32-4.17 (dd, —OCH₂—, 2H), 3.85-3.8 (m, —OCH_{A}CH_{B}CH₂—, 1H), 3.55-3.50 (m, —OCH_{A}CH_{B}CH₂—, 1H), 3.20 (s, —CH₂—, 1H), 3.12 (s, —CH₂—, 2H), 2.19 (q, —CH₂CH₃, 2H), 1.9-1.65 (m, —CH₂—, 2H), 1.65-1.45 (m, 2×—CH₂—, 4H), 1.15 (t, —CH₂CH₃, 3H) (FIG. 5).

Note: The crude was taken forward to the next deprotection reaction and the compound is purified in alcohol stage (PHR-108) using silica gel column chromatography.

### Stage-IV: Preparation of undeca-2,5,8-triyn-1-ol

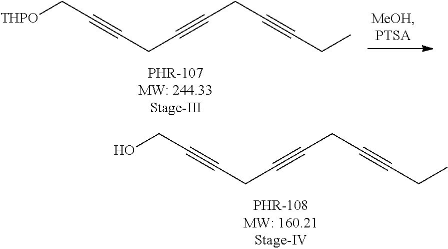

### Batch No #PAA-P-10-040-PHR-108-08

### Raw Materials:

| | | | | | | | Remarks |
|---|---|---|---|---|---|---|---|
| S. No. | Name of the Material | Qty. | | M. Wt. | Moles | Mole Ratio | (Source/CAS etc.,) |
| 1. | PHR-107 crude | 180 | g | 244.33 | 0.74 | 1.0 | 180 g of PHR-107 |
| | | | | | | | was obtained from |
| | | | | | | | 130 g of PHR-106. |
| 2. | p-Toluene sulfonic acid | 1.4 | g | 190.65 | 0.0074 | 0.01 | |
| 3. | Methanol | 900 | mL | — | — | 5.0 vol. | |

### Detailed Procedure:

1. Methanol (900 mL) was taken into a 2 L 4 neck RBF equipped with mechanical stirrer, reflux condenser, thermo pocket and heating provision.
2. PHR-107 (180 g) and PTSA (1.4 g) was added to the reaction mixture at 25 to 30° C.
3. The reaction temperature was raised to gentle reflux (65-70° C.) and maintained for 3 h.
Note: The reaction progress was monitored by TLC analysis (20% Ethyl acetate in hexanes) and visualizing spots using KMnO₄ and anisaldehyde stain. R_{f}=0.8 (PHR-107), 0.35 (PHR-108).
4. The reaction mixture was cooled to room temperature and the solvent was evaporated under vacuum at 50-55° C.
Observation: The crude was brown liquid.
5. The product was purified by column chromatography using silica gel (60-120 mesh) to obtain 58 g of pure PHR-108.

### Results

Weight of the product: 58 g
Yield: 50% (for two stages, from PHR-106)
Description: Pale yellow liquid
¹H NMR: complies with the structure.

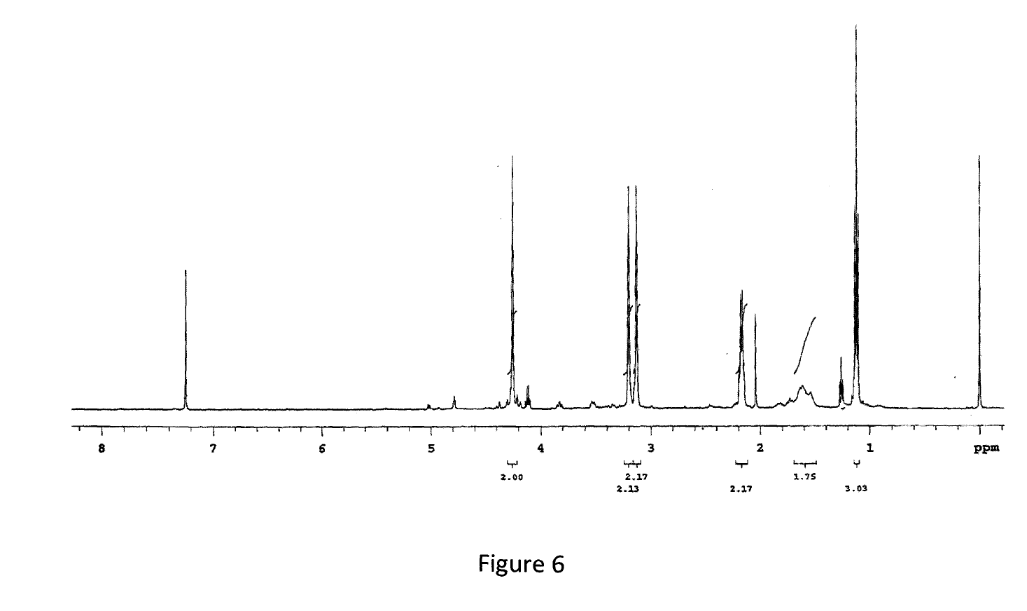

¹H NMR data: (CDCl₃, 500 MHz, ppm): 4.25 (s, —OCH₂—, 2H), 3.2 (s, —CH₂—, 2H), 3.1 (s, —CH₂—, 2H), 2.15 (q, —CH₂CH₃, 2H), 1.7-1.45 (bm, —OH, 1H), 1.15 (t, —CH₂CH₃, 3H) (FIG. 6).

### Stage-V: Preparation of undeca-2,5,8-triynyl 4-methylbenzenesulfonate

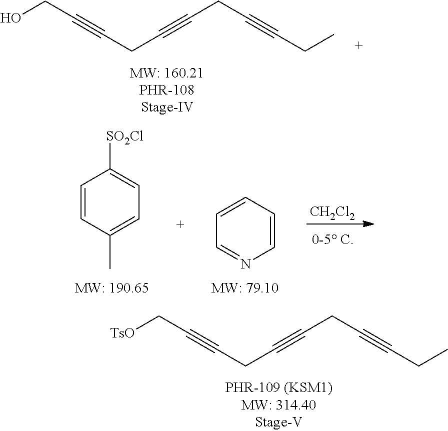

### Batch No #PAA-P-10-040-PHR-109-11

### Raw Materials:

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | PHR-108 | 25 | g | 160.21 | 0.16 | 1 | | |
| 2. | TsCl | 59.5 | g | 190.65 | 0.31 | 2 | | Commercial |
| 3. | Pyridine | 75 | mL | 79.1 | — | 3 | vol. | d = 0.98 |
| 4. | Dichloromethane (Lot-I) | 25 | mL | — | — | 1 | vol. | Commercial |
| 5. | Dichloromethane (Lot-II) | 87.5 | mL | — | — | 3.5 | vol. | Commercial |
| 6. | MTBE (Lot-I) | 500 | mL | — | — | 20 | vol. | Commercial |
| 7. | MTBE (Lot-II) | 250 | mL | — | — | 10 | vol. | Commercial |
| 6. | 3N Hydrochloric acid | 350 | mL | — | — | 14 | vol. | Commercial |
| 7. | 1M Hydrochloric acid | 250 | mL | — | — | 2 × 5 | vol. | |
| 8. | Saturated NaCl solution | 250 | mL | — | — | 10 | vol. | |

### Process Details:

1. Pyridine was charged in to a 1 L multi neck RBF, equipped with N₂ inlet, addition funnel and magnetic stirrer.
2. Reaction mixture was cooled to -5 to 0° C.
3. PHR-108 in dichloromethane (Lot-I) was added slowly to the reaction mixture over a period of 15 minutes at −5 to 0° C.
Observation: Dark brown color solution was observed.
4. The reaction mixture was stirred at −5° C. for 10 minutes.
5. Tosyl chloride in dichloromethane (Lot-II) was added to the reaction mixture over a period of 30 minutes at −5 to 0° C.
Observation: The reaction was found to be exothermic in nature.
Note: The addition of tosyl chloride was controlled so as to maintain the reaction temperature below 5° C.
6. Reaction mixture was stirred at the same temperature for 1 hour.
7. Reaction was monitored by TLC analysis.
Note: TLC analysis: 20% Ethyl acetate in hexanes and visualizing spots using KMnO₄ and anisaldehyde stain. R_{f}=0.4 (PHR-109), 0.35 (PHR-108).
8. Reaction mixture was quenched with 3N hydrochloric acid (350 mL).
Observation: Quenching was exothermic; the addition of HCl has to be controlled to keep the temperature under 10° C. During the process of quenching brown insoluble particles were formed.
9. The reaction mixture was diluted with MTBE (Lot-I) and stirred at 25 to 30° C. for 5 minutes.
10. The two layers were separated and the aqueous layer was extracted with MTBE (Lot-II).
11. The combined organic layer was washed with 1N hydrochloric acid (2×5 vol.).
12. The organic layer was washed with saturated NaCl solution (10 vol.).
13. The organic layer was dried over anhydrous sodium sulfate and evaporated under reduced pressure at 45° C.
Observation: PHR-109 was obtained as dark brown liquid.

### Results

Weight of the product: 32 g
Yield: 65%
Description: Brown to black liquid
¹H NMR: complies with the structure

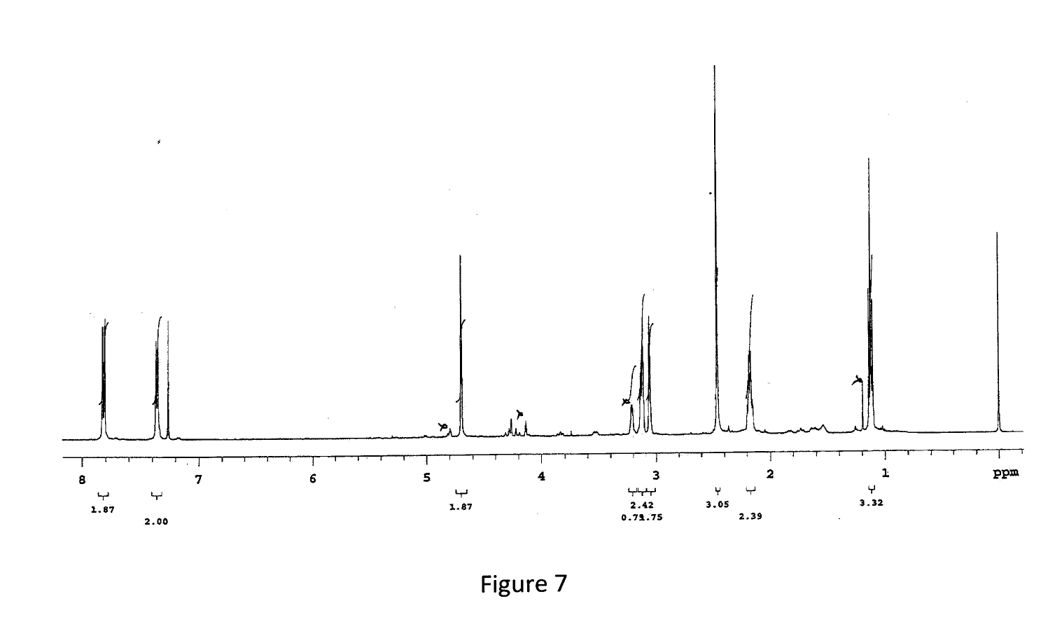

¹H NMR data: (CDCl₃, 500 MHz, ppm): 7.80 (d, ArCH, 2H), 7.35 (d, ArCH, 2H), 4.69 (s, —OCH₂—, 2H), 3.15-3.09 (s, —CH₂—, 2H), 3.09-3.02 (s, —CH₂—, 2H), 2.17 (q, —CH₂CH₃, 2H), 1.12 (t, —CH₂CH₃, 3H) (FIG. 7).

PHR-112 (KSM2): Preparation of methyl undeca-4,7,10-triynoate

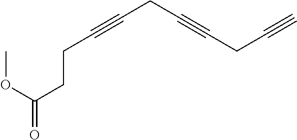

### Stage-I: Preparation of 4-hydroxybut-2-ynyl 4-methylbenzenesulfonate

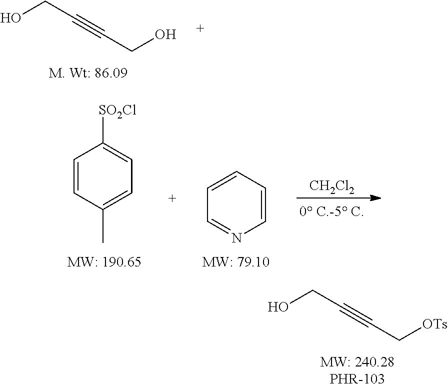

### Batch No #PAA-P-10-037-PHR-103-12

### Raw Materials:

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | 2-Butyne-1,4-diol | 1 | Kg | 86 | 11.62 | 1 | | AVRA Synthesis. |
| 2. | P-TsCl | 1.995 | Kg | 190.65 | 10.46 | 0.9 | | Commercial |
| 3. | Pyridine | 1.873 | L | 79 | 23.24 | 2 | | d = 0.98 |
| 4. | Dichloromethane (Lot-I) | 25 | L | — | — | 25 | vol. | Commercial |
| 5. | Dichloromethane (Lot-II) | 15 | L | — | — | 15 | vol. | Commercial |
| 6. | 3N HCl | 8 | L | — | 25.56 | 2.2 | | Commercial |
| 7. | 1N HCl | 10 | L | — | — | 10 | vol. | |
| 8. | Methanol | 3 | L | — | — | 3 | vol. | Commercial |
| 9. | Sat. NaCl solution | 5 | L | — | — | 5 | vol. | |

### Process Details:

1. Dichloromethane (25 L, lot-I) was charged in to a 100 L reactor.
2. 2-butyne1,4-diol (1 Kg) was charged in to the reactor.
Observation: 2-Butyne-1,4-diol was partially soluble in dichloromethane.
3. Reaction mixture was cooled to 0° C.
4. Pyridine was added to the reaction mixture over a period of 10 minutes at −5 to 0° C.
Observation: Clear solution.
5. Tosyl chloride in dichloromethane (Lot-II) was added to the reaction mixture over a period of 3 h at −5 to 0° C.
Observation: Exothermic reaction was observed, temperature controlled by slow addition of tosyl chloride.
6. Reaction mixture was stirred at the same temperature for 4 h.
Note: The reaction progress was monitored by spotting over an analytical silica gel TLC plate (50% Ethyl acetate in hexanes) and visualizing spots using KMnO₄ and anisaldehyde stain. R_{f}=0.3 (PHR-103), 0.1 (2-butyne-1,4-diol), 0.4 (ditosylated compound).
7. Reaction mixture was quenched with 3N hydrochloric acid
Note: Quenching was done below 10° C.
8. The two layers were separated.
9. The organic layer was washed with 1N hydrochloric acid.
10. The combined organic layer was washed with brine solution (5 L).
11. The organic layer was dried over anhydrous sodium sulfate and evaporated under reduced pressure at 45° C.
Observation: Dark brown liquid obtained.
12. The above dark brown liquid was taken in methanol (3 L).
13. The reaction mixture was cooled to 0-5° C.
Observation: White solids were crashed out from the methanolic solution and the solid was found to be exclusively ditosyl compound.
14. The suspended solids were filtered.
15. The filtrate was evaporated under reduced pressure at 45° C. to obtain PHR-103.
Observation: Brown oil

### Results

Weight of the product: 1.4 Kg
Yield: 50%
Description: Brown oil
¹H NMR Spectrum: Complies with the structure.

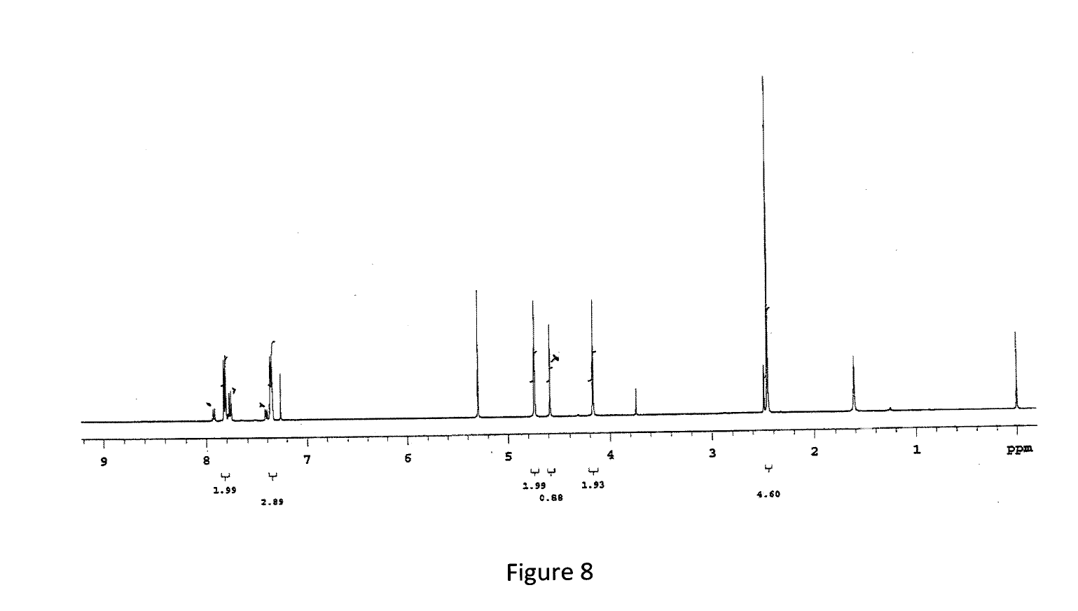

¹H NMR data: (CDCl₃, 500 MHz, ppm): 7.82 (d, ArCH, 2H), 7.34 (d, ArCH, 2H), 4.75 (s, —OCH₂—, 2H), 4.15 (s, —OCH₂—, 2H), 2.45 (s, ArCH₃, 3H) (FIG. 8).

### Stage-II: 6-(trimethylsilyl)hexa-2,5-diyn-1-ol

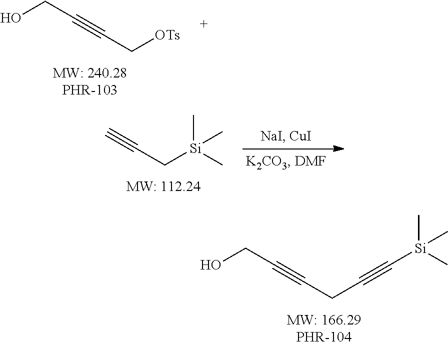

### Batch No #PAA-P-10-037-PHR-104-21

### Raw Materials:

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | PHR-103 | 450 | g | 240.28 | 1.87 | 1 | | |
| 2. | Trimethyl acetylene | 368 | g | 98.22 | 3.74 | 2 | | |
| 3. | CuI | 250 | g | 190.45 | 1.31 | 0.7 | | |
| 4. | NaI | 393 | g | 149.9 | 2.62 | 1.4 | | |
| 4. | K₂CO₃ | 362 | g | 138.2 | 2.62 | 1.4 | | |
| 5. | Dimethyl formamide | 3.15 | L | — | — | 7 | vol. | Commercial |
| | (Lot-I) | | | | | | | |
| 6. | Water (Lot-I) | 0.9 | L | — | — | 2 | vol. | |
| 7. | MTBE (Lot-I) | 1.8 | L | — | — | 4 | vol. | Commercial |
| 8. | MTBE (Lot-II) | 1.8 | L | — | — | 4 | vol. | Commercial |
| 9. | MTBE (Lot-III) | 1.8 | L | — | — | 4 | vol. | Commercial |
| 10. | Sat. NaCl | 1.8 | L | — | — | 4 | vol. | |
| 11. | Water | 3 × 1.8 | L | — | — | 3 × 4 | vol. | |

### Process Details:

1. DMF (Lot-I) was charged in to a 10 L 4 neck flask equipped with mechanical stirrer, argon bubbler, 1 L addition funnel and the solvent was degassed for 15 min.
2. CuI, sodium iodide and potassium iodide were charged in to the reactor.
Note: The reaction mixture turned in to yellow color with generation of heat (reaction mass was warmed to 30-35° C.
3. The reaction mixture was cooled to 0° C.
4. Trimethylsilyl acetylene was added to reaction mixture over 30 minutes.
5. The reaction mixture was stirred at 0-5° C. for 30 minutes maintaining the degassing conditions.
6. PHR-103 was added in to reaction mixture over 30 minutes and the degassing was continued for another 30 min.
7. The argon flow was slightly reduced so as to maintain inert atmosphere in the reaction flask and the stirring was continued for 18 hours.
Note: The reaction progress was monitored by TLC analysis (20% Ethyl acetate in hexanes and visualizing spots using anisaldehyde solution). R_{f}=0.33 (PHR-104).
8. MTBE (Lot-I) and water (Lot-I) was added to the reaction mixture and stirred for 15 minutes.
9. The reaction mixture was filtered on Celite™ pad and washed with MTBE (lot-II)
10. The layers of filtrate were separated and the aqueous layer was extracted with MTBE (Lot-III).
11. The combined organic layer was washed with water (3×1.8 L; until there are no solid precipitation observed) and with saturated NaCl (1×1.8 L).
12. The organic layer was dried over anhydrous sodium sulfate and concentrated on rotovap to obtain the crude PHR-110.
13. The crude was purified by silica gel column chromatography using 60-120 mesh silica and the compound was eluted in 5% ethyl acetate in hexanes.

### Results

Weight of the product: 171 g of crude.
Yield: 55%
Description: Brown to black liquid
¹H NMR: complies with the structure

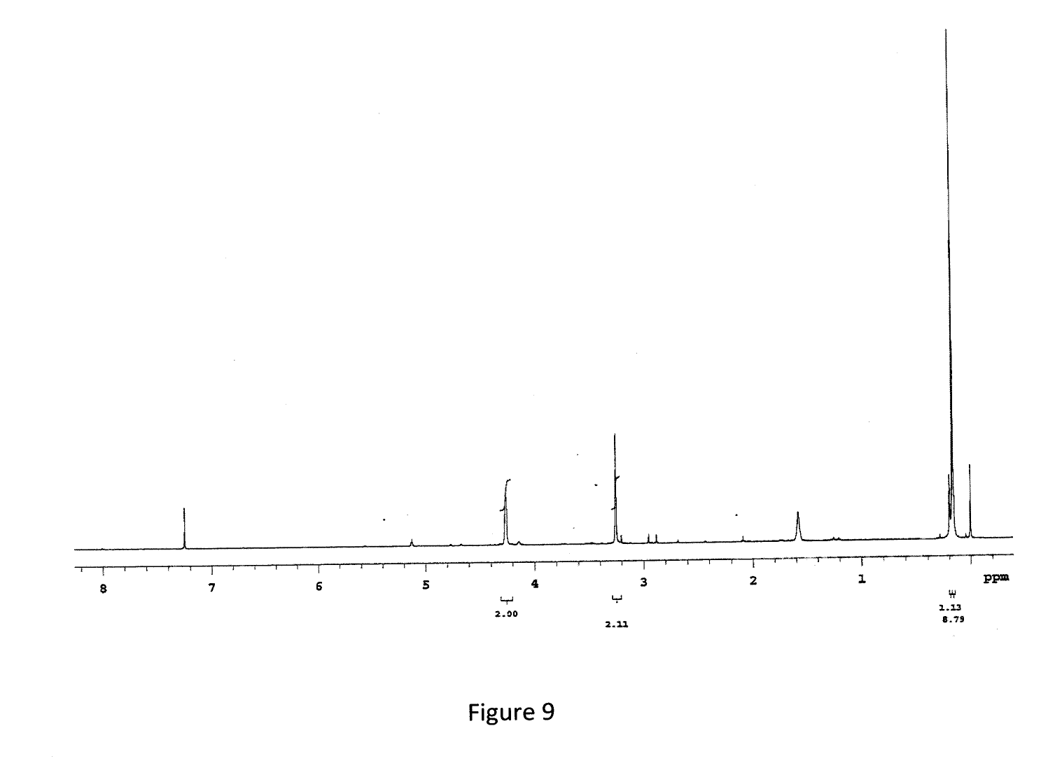

¹H NMR data: (CDCl₃, 500 MHz, ppm): 4.26 (s, —OCH₂—, 2H), 3.27 (s, —CH₂—, 2H), 0.18 (s, 3×—CH₃, 9H) (FIG. 9).

### Stage-III: Preparation of 6-(trimethylsilyl)hexa-2,5-diynyl 4-methyl benzene sulfonate

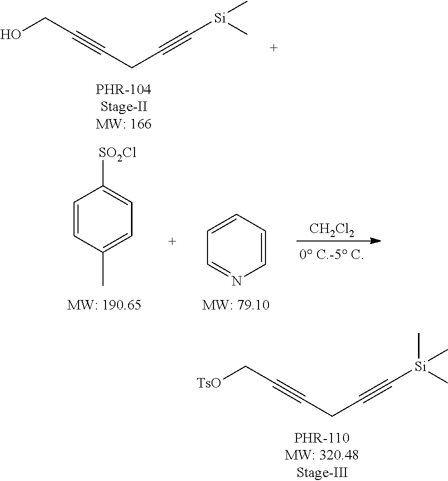

### Batch No #PAA-P-10-037-PHR-110-19

### Raw Materials:

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | PHR-104 | 40 | g | 166 | 0.1807 | 1 | | |
| 2. | Tosyl chloride | 92 | g | 190.65 | 0.3615 | 2 | | Commercial |
| 3. | pyridine | 120 | mL | — | — | 3 | vol. | Commercial |
| 4. | Dichloromethane (Lot-I) | 40 | mL | — | — | 1 | vol. | Commercial |
| 5. | Dichloromethane (Lot-II) | 140 | mL | — | — | 3.5 | vol. | Commercial |
| 6 | MTBE (Lot-I) | 360 | mL | — | — | 9 | vol. | Commercial |
| 7. | MTBE (Lot-II) | 180 | mL | — | — | 4.5 | vol. | Commercial |
| 8. | 3N Hydrochloric acid | 560 | mL | — | — | | | Commercial |
| 9. | 1N Hydrochloric acid | 480 | mL | — | — | 12 | vol. | |
| 10. | Saturated Sodium | 240 | mL | — | — | 6 | vol. | |
| | chloride solution | | | | | | | |

| | | | | | |
|---|---|---|---|---|---|
| 11. | anhydrous sodium | As | — | — | — |
| | sulfate | needed | | | |

### Process Details:

1. Pyridine was charged in to a 1 L 4 neck RBF equipped with N₂ inlet, addition funnel and a magnetic stirrer.
2. The reaction mixture was cooled to −5° C.
3. PHR-104 (40 g) in dichloromethane (Lot-I) was added slowly to the reaction mixture over a period of 15 minutes at −5 to 0° C.
Observation: Dark brown color solution was observed.
4. The reaction mixture was stirred at −5° C. for 10 minutes.
5. Tosyl chloride (92 g) in dichloromethane (Lot-II) was added to the reaction mixture over a period of 30 minutes at −5 to 0° C.
Observation: The reaction being exothermic, the temperature of the reaction was maintained by the rate of tosyl chloride addition.
6. Reaction mixture was stirred at the same temperature for 1 h.
Note: The reaction progress was monitored by TLC analysis (20% Ethyl acetate in hexanes and visualizing spots using anisaldehyde solution). R_{f}=0.4 (PHR-110), 0.33(PHR-104).
7. Reaction mixture was quenched with 3N hydrochloric acid.
Note: The quenching was done under 10° C.
8. The reaction mixture was diluted with MTBE (Lot-I) and stirred at RT for 5 minutes.
Observation: Dark brown clear solution was observed.
9. The two layers were separated.
10. The aqueous layer was again extracted with MTBE (Lot-II).
11. The combined organic layer was washed with 1N Hydrochloric acid (2×6vol.).
12. The organic layer was washed with brine solution (6 vol.).
13. The organic layer was dried over anhydrous sodium sulfate and evaporated under reduced pressure at 45° C.
Observation: Dark brown liquid obtained.

### Results

Weight of the product: 60 g
% of yield: 78%
Description: Brown oil
¹H NMR Spectrum: Complies with the structure.

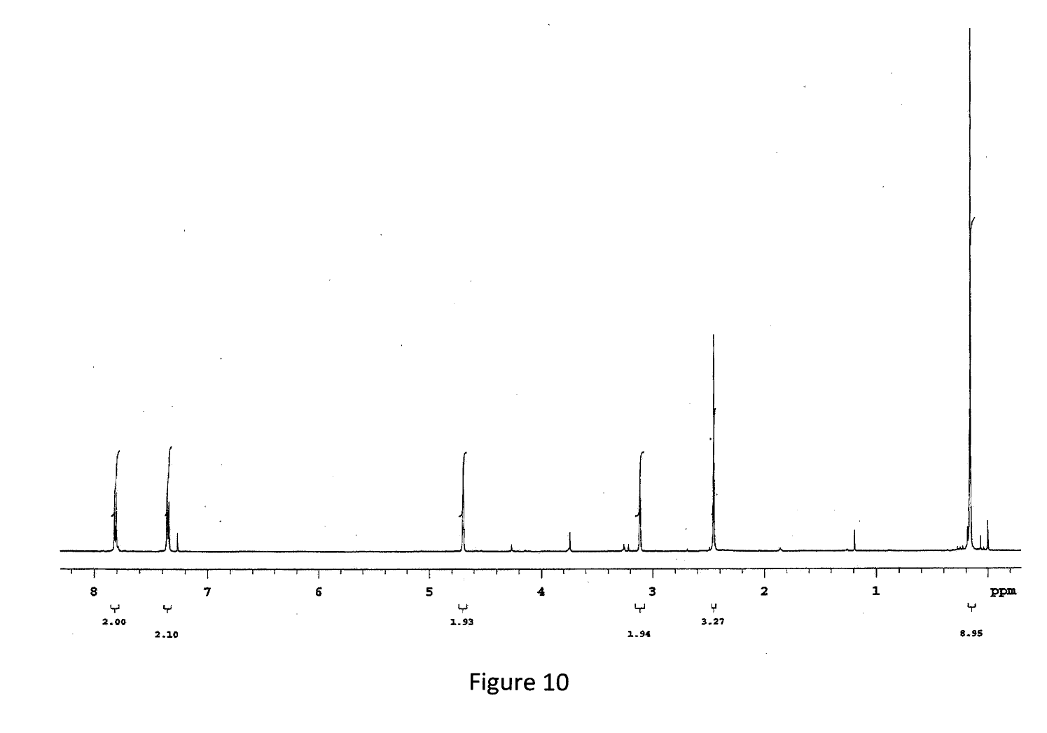

¹H NMR data: (CDCl₃, 500 MHz, ppm): 7.81 (d, ArCH, 2H), 7.35 (d, ArCH, 2H), 4.70 (s, —OCH₂—, 2H), 3.12 (s, —CH₂—, 2H), 2.45 (s, ArCH₃, 3H), 0.16 (s, 3×—CH₃, 9H) (FIG. 10).

### Stage-III: Preparation of methyl 11-(trimethylsilyl)undeca-4,7,10-triynoate

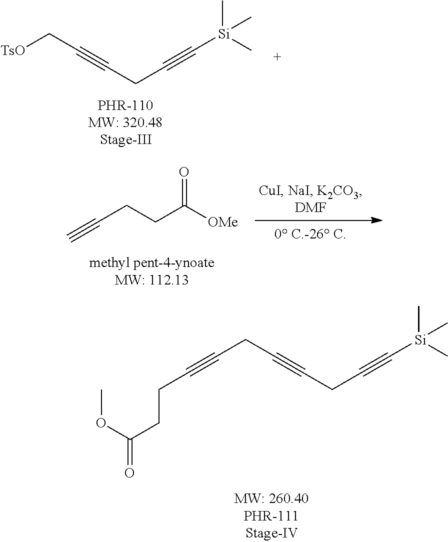

### Batch No #PAA-P-10-037-PHR-111-020

### Raw Materials:

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | Methyl-4-pentynoate | 20 | g | 112.13 | 0.178 | 1 | | Prepared from acid |
| 2. | PHR-110 | 80 | g | 320.38 | 0.249 | 1.4 | | |
| 3. | CuI | 23.7 | g | 190.45 | 0.124 | 0.7 | | |
| 4. | NaI | 37.35 | g | 149.9 | 0.249 | 1.4 | | |
| 5. | K₂CO₃ | 34.44 | g | 138.2 | 0.249 | 1.4 | | |
| 6. | Dimethylformamide (Lot-I) | 100 | mL | — | — | 5 | vol. | LR Grade |
| 7. | Dimethylformamide (Lot-I) | 20 | mL | — | — | 1 | vol. | LR Grade |
| 8. | MTBE (Lot-I) | 500 | mL | — | — | 25 | vol. | Commercial |
| 9. | Water (Lot-I) | 20 | mL | — | — | 1 | vol. | Commercial |
| 10. | Water (Lot-II) | 200 | mL | — | — | 10 | vol. | |
| 11. | Water (Lot-III) | 200 | mL | — | — | 10 | vol. | |
| 12. | Sat. NaCl solution | 100 | mL | | | 5 | vol. | |

### Process Details:

1. DMF (100 mL, 5 vol.) was charged in to a 500 mL 4 neck flask equipped with mechanical stirrer, argon bubbler and 250 mL addition funnel and the solvent was degassed for 15 min.
2. CuI, Sodium iodide and potassium iodide were charged in to the flask.
Note: The reaction mass turned in to yellow color with generation of heat (reaction mass was warmed to 30-35° C.
3. The reaction mass was chilled to 0° C. using ice/water bath.
4. Methyl-4-pentynoate was added to reaction mass over 5 min.
5. The reaction mixture was stirred at 0-5° C. for 15 minutes maintaining the degassing conditions.
6. Using addition funnel, PHR-110 was added in to reaction mass over 10 minutes and the degassing was continued for another 30 min.
7. The argon flow was slightly reduced so as to maintain inert atmosphere in the reaction flask and the stirring was continued for 18 hours.
Note: The reaction progress was monitored by TLC analysis (20% Ethyl acetate in hexanes and visualizing spots using anisaldehyde solution). R_{f}=0.5 (PHR-110) and 0.55 (PHR-111).
8. To the reaction mass MTBE (Lot I) and water (Lot I) were added and stirred for 15 minutes.
9. The reaction mixture was filtered on Celite™ bed and washed with MTBE (Lot-II).
10. The filtrate layers were separated and the organic layer was washed with water (Lot-II and III).
11. The organic layer was finally washed with brine and dried over anhydrous sodium sulfate.
12. The organic layer was concentrated under reduced pressure to obtain the crude PHR-111.

### Results:

Weight of the product: 21 g of crude.
Yield: 45%
Description: Brown to black liquid
¹H NMR: complies with the structure

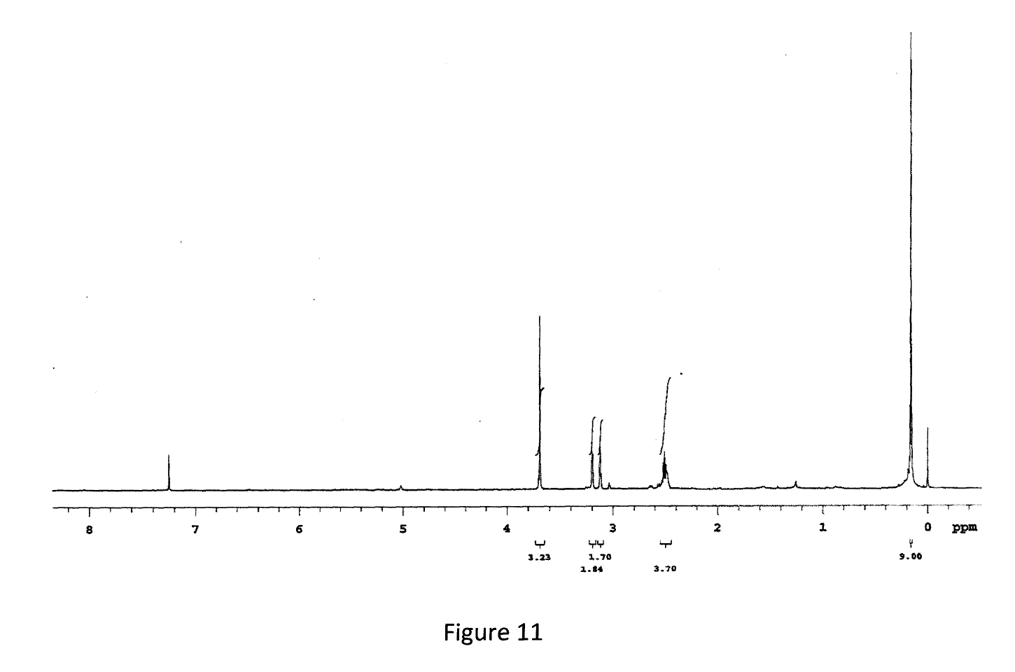

¹H NMR data: (CDCl₃, 500 MHz, ppm): 3.69 (s, —OCH₃, 3H), 3.19 (s, —CH₂—, 2H), 3.12 (s, —CH₂—, 2H), 2.55-2.25 (m, 2×—CH₂—, 4H), 0.18 (s, 3×—CH₃, 9H) (FIG. 11).

### Stage-V: Preparation of methyl undeca-4,7,10-triynoate

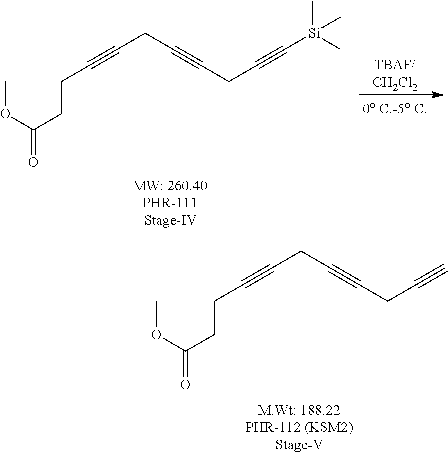 [SMILES: COC(=O)CCC#CCC#CCC#C[Si](C)(C)C ; C#CCC#CCC#CCCC(=O)OC]

### Batch No #PAA-P-10-037-PHR-112-22

### Raw Materials:

| | | | | | | | Remarks |
|---|---|---|---|---|---|---|---|
| S. No. | Name of the Material | Qty. | | M. Wt. | Moles | Mole Ratio | (Source/CAS etc.,) |
| 1. | PHR-111 | 16 | g | 260.4 | 0.0615 | 1 | |
| 2. | TBAF | 9.69 | g | 315.5 | 0.0307 | 0.5 | |
| 3. | Dichloromethane | 480 | mL | — | — | 30 vol. | |
| 4. | Ice-cold water | 320 | mL | — | — | 20 vol. | |
| 5. | Water | 320 | mL | — | — | 20 vol. | |
| 6. | Sat. NaCl solution | 320 | mL | — | — | 20 vol. | |

### Process Details:

1. Dichloromethane (480 mL) and PHR-111 (16 g) were charged into a 1 L 4 neck round bottom flask.
2. Reaction mixture was cooled to below 0° C. (−5 to 0° C.)
3. TBAF (9.69 g) was added to the reaction mixture over a period of 30 minutes in 10 lots maintaining the reaction temperature −5 to 0° C.
4. Reaction mixture was stirred for 1 h at −5 to 0° C.
Note: The reaction progress was monitored by TLC analysis (10% Ethyl acetate in hexanes and visualizing spots using anisaldehyde solution). R_{f}=0.35 (PHR-112), 0.4 (PHR-111).
5. Reaction mixture was quenched with ice-chilled water
6. The two layers were separated.
7. The organic layer was washed with water and then saturated sodium chloride solution.
8. The organic layer was dried over sodium sulfate and evaporated under reduced pressure to afford PHR-112 as black liquid.
9. The crude was purified by silica gel column chromatography using 60-120 mesh silica and the product was eluted in 3% ethyl acetate in hexanes.

### Results

Weight of the product: 8 g of crude.
Yield: 69%
Description: Brown to black liquid
¹H NMR: complies with the structure

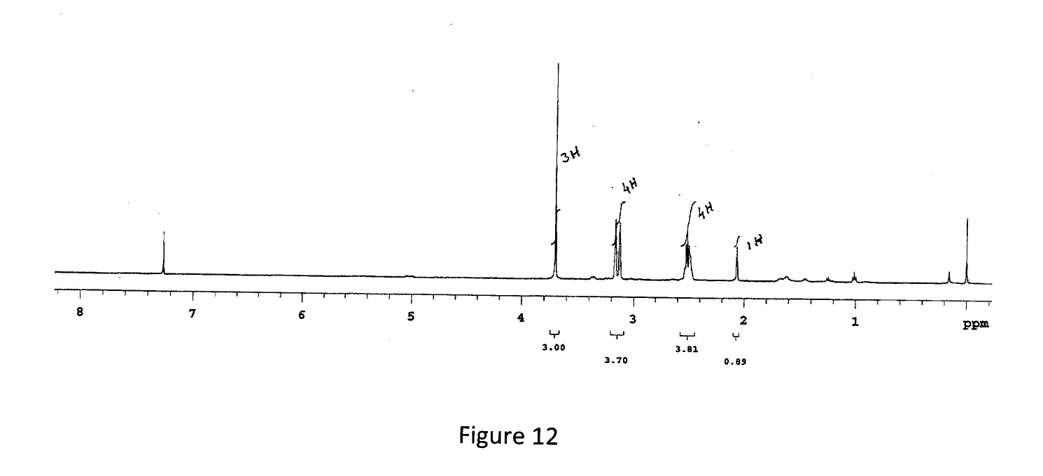

¹H NMR data: (CDCl₃, 500 MHz, ppm): 3.72(s, —OCH₃, 3H), 3.18 (s, —CH₂—, 2H), 3.12 (s, —CH₂—, 2H), 2.55-2.42 (m, 2×—CH₂—, 4H), 2.05 (s, 1H) (FIG. 12).

### Coupling of KSM1 and KSM2:

### Preparation of methyl docosa-4,7,10,13,16,19-hexaynoate (PHR-114)

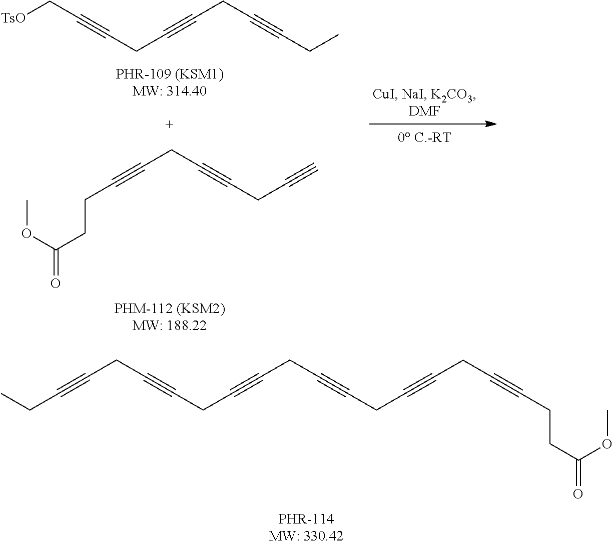 [SMILES: CCC#CCC#CCC#CCOS(=O)(=O)c1ccc(C)cc1 ; C#CCC#CCC#CCCC(=O)OC ; CCC#CCC#CCC#CCC#CCC#CCC#CCCC(=O)OC]

### Batch No #PAA-P-10-037-PHR-114-23

### Raw Materials:

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | PHR-112 | 10 | g | 188.22 | 0.053 | 1 | | |
| 2. | PHR-109 | 23.4 | g | 314.40 | 0.075 | 1.4 | | |
| 3. | CuI | 7.1 | g | 190.45 | 0.037 | 0.7 | | |
| 4. | NaI | 11.2 | g | 149.9 | 0.075 | 1.4 | | |
| 5. | K₂CO₃ | 10.3 | g | 138.2 | 0.075 | 1.4 | | |
| 6. | Dimethylformamide (Lot-I) | 60 | mL | — | — | 6 | vol. | LR Grade |
| 7. | Dimethylformamide (Lot-I) | 60 | mL | — | — | 6 | vol. | LR Grade |
| 8. | MTBE (Lot-I) | 250 | mL | — | — | 25 | vol. | Commercial |
| 9. | MTBE (Lot-II) | 250 | mL | — | — | 25 | vol. | Commercial |
| 10. | MTBE (Lot-III) | 250 | mL | — | — | 25 | vol. | Commercial |
| 11. | Water (Lot-I) | 10 | mL | — | — | 1 | vol. | |
| 12. | Water (Lot-II) | 100 | mL | — | — | 10 | vol. | |
| 13. | Water | 100 | mL | — | — | 10 | vol. | |
| 14. | Sat. NaCl solution | 50 | mL | | | 5 | vol. | |

Process Details:

The reaction was performed under same protocol as developed for PHR-111.
Note: The reaction progress was monitored by TLC analysis (10% Ethyl acetate in hexanes and, visualizing spots using anisaldehyde solution). R_{f}=0.38 (PHR-114), 0.45 (PHR-112).

### Results

Weight of the product: 6 g.
Yield: 33%
Description: yellow to brown solid
¹H NMR: complies with the structure

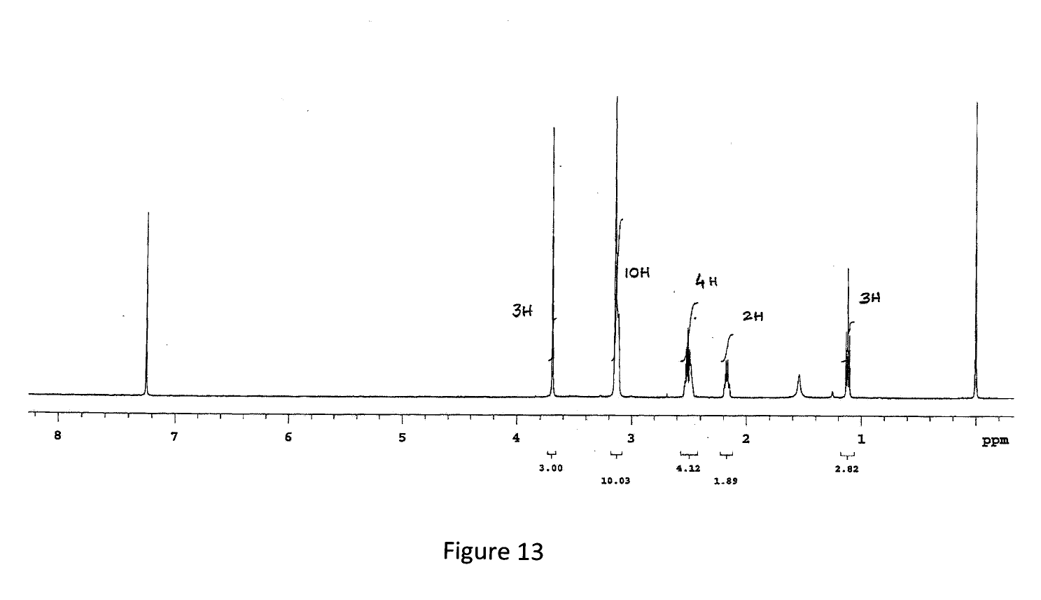

¹H NMR data: (CDCl₃, 500 MHz, ppm): 3.70 (s, —OCH₃, 3H), 3.18-3.1 (m, 5 x —CH₂—,10H), 2.55-2.42 (m, 2×—CH₂—, 4H), 2.15 (q, —CH₂CH₃, 2H), 1.13 (t, —CH₂CH₃, 2H) (FIG. 13).

### Preparation of (4Z,7Z,10Z,13Z,16Z,19Z)-methyl docosa-4,7,10,13,16,19-hexaenoate (PHR-115)

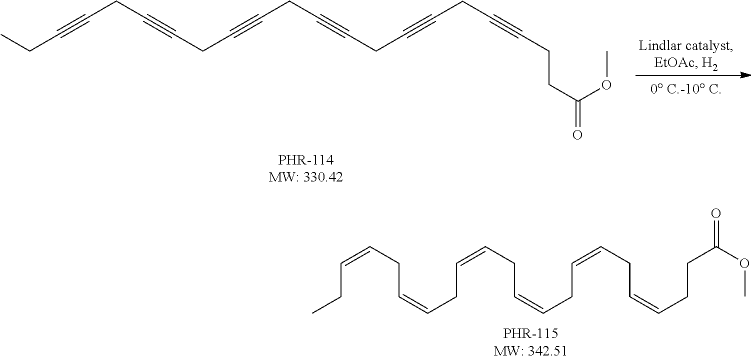

Batch No. PAA-P-10-037-PHR-115-44:

### Raw Materials:

| | | | | | | | | Remarks |
|---|---|---|---|---|---|---|---|---|
| S. No. | Name of the Material | Qty. | | M. Wt. | Moles | Mole Ratio | | (Source/CAS etc.,) |
| 1. | PHR-114 | 500 | mg | 330.42 | 1.51 mmol | 1 | | |
| 2. | Lindlar's Catalyst | 500 | mg | — | — | 100% | w/w | Aldrich |
| 3. | Quinoline | 50 | μL | | | 0.1 | vol. | |
| 4. | Ethanol | 41.2 | mL | — | — | 82.4 | vol. | |

Process Details:
1. Ethanol (41.2 mL) and PHR-114 (500 mg) were charged in to a 100 mL multi neck round bottom flask.
2. Lindlar's catalyst (500 mg, 100% w/w) was then charged in to the reaction flask at room temperature.
3. Quinoline (50 μL, 0.1 vol.) was charged in to reaction mixture using a micro syringe.
4. The reaction flask was repeatedly (3 times) purged with hydrogen gas using hydrogen balloon and the reaction mixture was stirred at room temperature.
5. The reaction was monitored by TLC (2×5% ethyl acetate in hexanes, R_{f}=0.55, anisaldehyde stain).
6. The reaction was stopped by removing hydrogen atmosphere, when the starting material was almost consumed.
7. The reaction mixture was passed through Celite™ and the catalyst was washed with ethanol (2×10 vol.).
8. Ethanol was removed on rotary evaporator, to obtain crude product (530 mg).
9. Column chromatography was done using 5% ethyl acetate in hexanes as mobile phase to obtain pure PHR-115 (120 mg, 23% yield).

### p Results

Weight of the product: 120 mg
Yield: 23%
Description: N/A
¹H NMR: complies with the structure

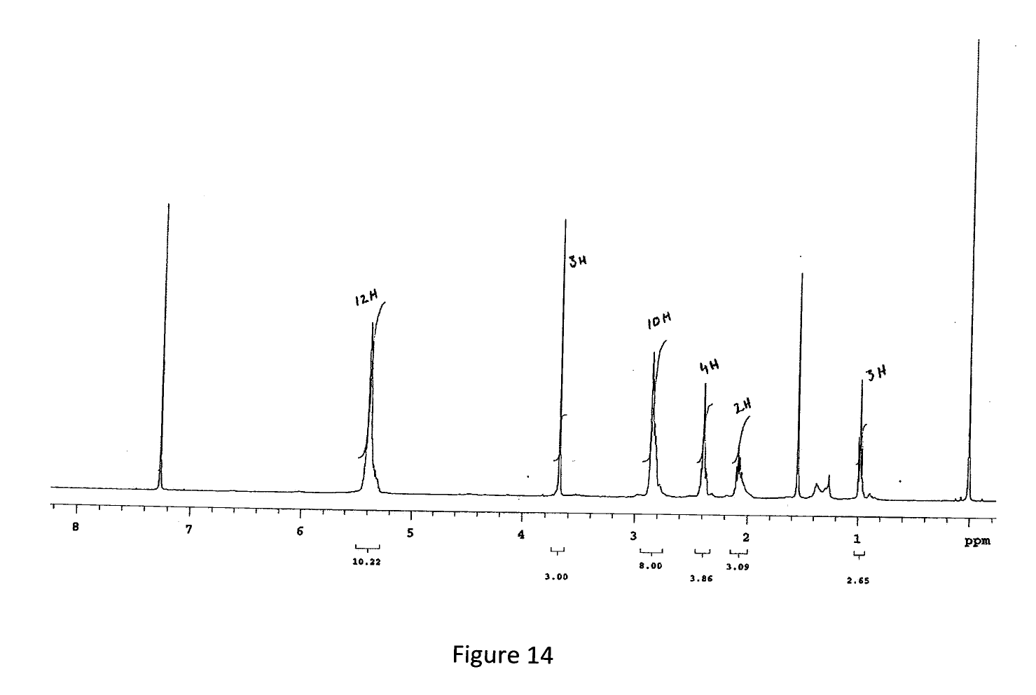

¹H NMR data: (CDCl₃, 500 MHz, ppm): 5.4-5.3 (m, 12H), 3.65 (s, —OCH₃, 3H), 2.9-2.8 (m, 5×—CH₂—, 10H), 2.41-2.35 (m, 2×—CH₂—, 4H), 2.12-2.0 (m, —CH₂CH₃, 2H), 0.95 (t, —CH₂CH₃, 3H) (FIG. 14).

### Preparation of (4Z,7Z,10Z,13Z,16Z,19Z)-docosa-4,7,10,13,16,19-hexaenoic acid (DHA)

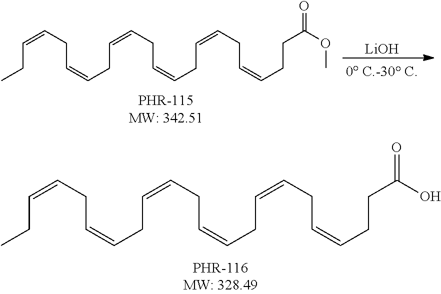 [SMILES: CC/C=C\C/C=C\C/C=C\C/C=C\C/C=C\C/C=C\CCC(=O)OC ; "LiOH" ; CC/C=C\C/C=C\C/C=C\C/C=C\C/C=C\C/C=C\CCC(=O)O]

### Batch No #PAA-P-10-37-PHR-116-48

### Raw Materials:

| | | | | | | | | Remarks |
|---|---|---|---|---|---|---|---|---|
| S. No. | Name of the Material | Qty. | | M. Wt. | Moles | | Mole Ratio | (Source/CAS etc.,) |
| 1. | PHR-115 | 140 | mg | 342.5 | 0.409 | mmol | 1 | |
| 2. | Lithium Hydroxide | 56 | mg | —42 | —1.2 | mmol | 3.0 | Aldrich |

| | | | | | | | |
|---|---|---|---|---|---|---|---|
| 3. | THF:H₂O (3:1 mixture) | 4 | mL | — | — | — | |
| 4. | 1M citric acid | 5 | mL | — | — | 82.4 vol. | Chemlabs |
| 5. | Diethyl ether | 20 | mL | — | — | — | Finar |

Process Details:
1. THF:H₂O (3:1mixture) and PHR-115 (140 mg) were charged in to a 50 mL multi neck round bottom flask.
2. Cooled the reaction mixture temperature to 0° C., added lithium hydroxide (56 mg) and allowed the temperature to stir at RT for 12 h.
3. The reaction was monitored by TLC (20% Ethyl acetate in hexanes, R_{f}=0.1, anisaldehyde stain).
4. The reaction was cooled to 0° C. and slowly quenched with aqueous citric acid (until pH was ˜4; 5 mL) and extracted with diethyl ether (2×4 vol.).
5. The ether layer was washed with water (1×3 vol.), brine solution (1×3 vol.) and dried over anhydrous sodium sulphate.
6. Ether layer was removed on rotary evaporator, to obtain crude product (100 mg).

### Results

Weight of the product: 100 mg.
Yield: 74.6%
Description: N/A
¹H NMR: complies with the structure.

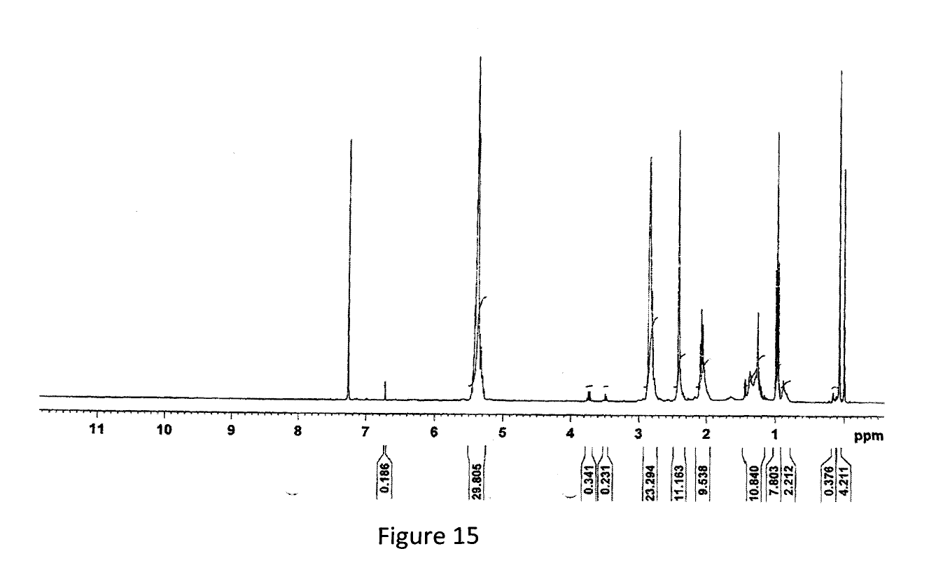

¹H NMR data: (CDCl₃, 400 MHz, ppm): 5.5-5.2 (m, 12H), 2.9-2.7 (m, 5×—CH₂—, 10H), 2.45-2.3 (m, 2×—CH₂—, 4H), 2.1-1.9 (m, —CH₂CH₃, 2H), 0.95 (t, —CH₂CH₃, 3H) (FIG. 15).

One or more currently preferred embodiments have been described by way of example. It will be apparent to persons skilled in the art that a number of variations and modifications can be made without departing from the scope of the invention as defined in the claims.

1. A compound represented by Formula II: 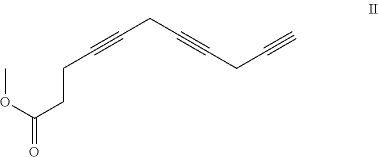
or a pharmaceutically acceptable salt or stereoisomer thereof

2-3. (canceled)

4. A method for preparing a compound represented by Formula I 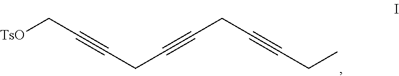 [SMILES: CCC#CCC#CCC#CCOS(=O)(=O)c1ccc(C)cc1]
the method comprising the steps of:
(a) converting a propargyl alcohol to a compound represented by Formula IV: 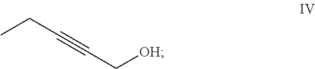
(b) converting the compound represented by Formula IV to a compound represented by Formula V: 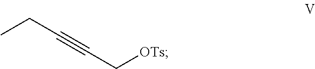 [SMILES: CCC#CCOS(=O)(=O)c1ccc(C)cc1]
(c) converting propargyl alcohol to a compound represented by Formula VIa: 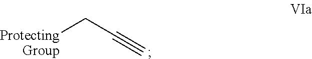
(d) converting the compound represented by Formula VIa to a compound represented by Formula VIIa: 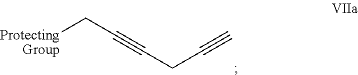
(e) coupling the compound represented by Formula VIIa with the compound represented by Formula V to yield a compound represented by Formula VIIIa: 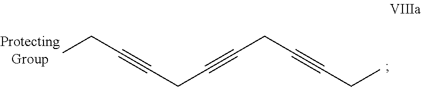
(f) converting the compound represented by Formula VIIIa to a compound represented by Formula IX: 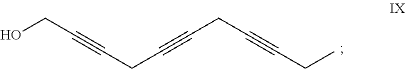
and
(g) converting the compound represented by Formula IX to the compound represented by Formula I.

5. A method as claimed in claim 4, wherein step (a) comprises reacting said propargyl alcohol with an ethyl halide in the presence of a strong base and a polar aprotic solvent to obtain the compound represented by Formula IV.

6. A method as claimed in claim 5, wherein the ethyl halide is ethyl iodide, the strong base is n-BuLi, and the polar aprotic solvent is HMPA in THF.

7. A method as claimed in claim 6, wherein the step (a) is carried out at a temperature of between −78° C. to room temperature.

8. A method as claimed in claim 4, wherein the step (b) comprises reacting the compound represented by Formula IV with 4-Toluenesulfonyl chloride (TsCl) and a strong inorganic base to obtain the compound represented by Formula V.

9. A method as claimed in claim 8, wherein the strong inorganic base is potassium hydroxide (KOH).

10. A method as claimed in claim 4, wherein the step (c) comprises reacting propargyl alcohol with an alcohol protecting agent or group to obtain a compound represented by Formula VIa.

11. A method as claimed in claim 10, wherein the alcohol protecting agent or group is dihydropyran (DHP) and the DHP protection reaction produces a tetrahydropyranyl ether according to the compound represented by Formula VI:

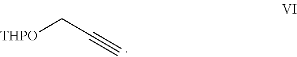

12. A method as claimed in claim 4, wherein the step (d) comprises reacting the magnesium acetylide compound represented by Formula VIa with a propargyl halide in the presence of a catalyst to obtain the compound represented by Formula VIIa.

13. A method as claimed in claim 12 wherein the propargyl halide is propargyl bromide and the catalyst is CuCl.

14. A method as claimed in claim 4, wherein the coupling reaction of step (e) is conducted in the presence of a polar aprotic solvent to obtain the compound represented by Formula VIIIa.

15. A method as claimed in claim 14, wherein the polar aprotic solvent is dimethylformamide (DMF).

16. A method as claimed in claim 4, wherein the step (f) comprises deprotecting the compound represented by Formula VIIIa in the presence of p-TSA/methanol.

17. A method as claimed in claim 16, wherein the deprotecting is carried out at a temperature of about 60° C.

18. A method as claimed in claim 4, wherein the step (g) comprises reacting the compound represented by Formula IX with a tosyl halide in pyridine to obtain the compound represented by Formula I.

19. A method as claimed in claim 18, wherein the tosyl halide is tosyl chloride.

20. A method for preparing a compound represented by Formula II: 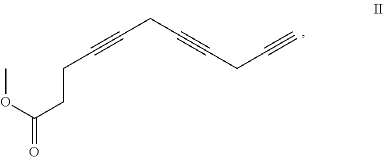
the method comprising the steps of:
(h) converting 2-Butyne-1,4-diol to a compound represented by Formula X: 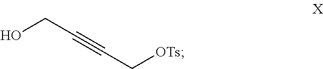 [SMILES: Cc1ccc(S(=O)(=O)OCC#CCO)cc1]
(i) converting the compound represented by Formula X to a compound represented by Formula XI: 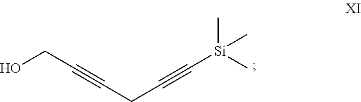
(j) converting the compound represented by Formula XI to a compound represented by Formula XII: 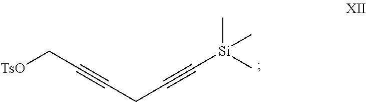 [SMILES: Cc1ccc(S(=O)(=O)OCC#CCC#C[Si](C)(C)C)cc1]
(k) converting the compound represented by Formula XII to a compound represented by Formula XIII: 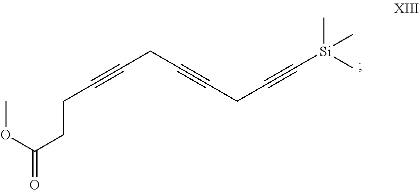
and
(l) converting the compound represented by Formula XIII to the compound represented by Formula II.

21. A method as claimed in claim 20, wherein the step (a) comprises reacting the 2-butyne-1,4-diol with tosyl chloride and pyridine in an organic solvent to obtain the compound represented by Formula X.

22. A method as claimed in claim 21, wherein the organic solvent is dichloromethane.

23. A method as claimed in claim 20, wherein the step (b) comprises reacting the compound represented by Formula X with trimethylsilyl acetylene to produce the compound represented by Formula XI.

24. A method as claimed in claim 20, wherein the step (c) comprises tosylation of the compound represented by Formula XI to produce the compound represented by Formula XII.

25. A method as claimed in claim 24, wherein the tosylation reaction is carried out by reacting the compound represented by Formula XI with p-toluenesulfonic acid.

26. A method as claimed in claim 20, wherein the step (d) comprises reacting the compound represented by Formula XII with methyl-4-pentynoate to produce the compound represented by Formula XIII.

27. A method as claimed in claim 20, wherein the step (e) comprises deprotecting the compound represented by Formula XIII with tetra-n-butylammonium fluoride (TBAF) in a solvent to produce the compound represented by Formula II.

28. A method as claimed in claim 27, wherein the solvent is dichloromethane.

29. A method of preparing DHA: 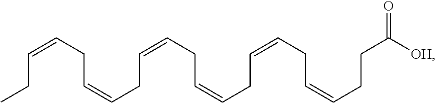 [SMILES: CC/C=C\C/C=C\C/C=C\C/C=C\C/C=C\C/C=C\CCC(=O)O]
the method comprising the steps of:
(m) coupling a compound represented by Formula I: 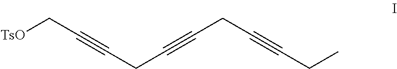 [SMILES: CCC#CCC#CCC#CCOS(=O)(=O)c1ccc(C)cc1]
with a compound represented by Formula II: 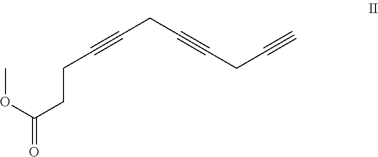
to obtain a compound represented by Formula III: 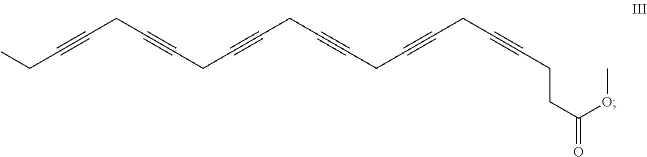 [SMILES: CCC#CCC#CCC#CCC#CCC#CCC#CCCC(=O)OC]
(n) partially hydrogenating the compound represented by Formula III to produce a compound represented by Formula XIV: 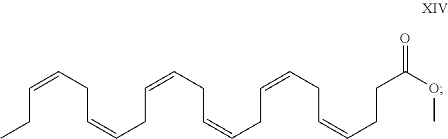 [SMILES: CC/C=C\C/C=C\C/C=C\C/C=C\C/C=C\C/C=C\CCC(=O)OC]
and
(o) hydrolysing the compound represented by Formula XIV to produce DHA,
or, the method comprising the steps of:
(a′) partially hydrogenating a compound represented by Formula III: 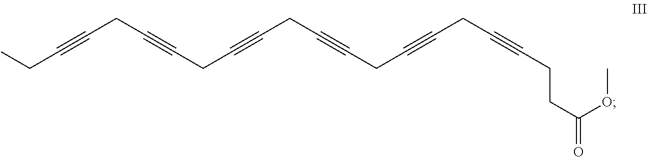 [SMILES: CCC#CCC#CCC#CCC#CCC#CCC#CCCC(=O)OC]
to produce a compound represented by Formula XIV: 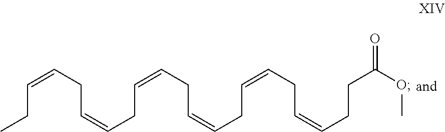 [SMILES: CC/C=C\C/C=C\C/C=C\C/C=C\C/C=C\C/C=C\CCC(=O)OC]
(b′) hydrolysing the compound represented by Formula XIV to produce DHA.

30. A method as claimed in claim 29, wherein the step (a) is conducted in the presence of CuI, NaI and K₂CO₃ in a polar aprotic solvent.

31. A method as claimed in claim 30, wherein the polar aprotic solvent is dimethylformamide (DMF).

32. A method as claimed in claim 29, wherein the step (b) or (a′) is conducted in the presence of Lindlar's catalyst.

33-34. (canceled)
